(19) United States
(12) Patent Application Publication
(10) Pub. No.: US 2022/0202712 A1
(43) Pub. Date: Jun. 30, 2022

(54) SELF-EMULSIFYING DRUG DELIVERY SYSTEMS FOR DELIVERY OF LIPOPHILIC COMPOUNDS

(71) Applicant: Yissum Research Development Company of the Hebrew University of Jerusalem Ltd., Jerusalem (IL)

(72) Inventors: Simon Benita, Tel Aviv (IL); Taher Nassar, Kfar Tur'an (IL)

(21) Appl. No.: 17/604,183

(22) PCT Filed: Apr. 7, 2020
(86) PCT No.: PCT/IL2020/050424

§ 371 Date: Oct. 15, 2021

Related U.S. Application Data
(60) Provisional application No. 62/835,878, filed on Apr. 18, 2019.

(51) Int. Cl. A61K 9/107; A61K 47/10; A61K 9/00; A61K 36/185

(52) U.S. Cl. CPC A61K 9/1075; A61K 47/10; B82Y 5/00; A61K 36/185; A61K 9/0053

(57) ABSTRACT

The present disclosure provides self-emulsifying drug delivery systems for delivery of lipophilic compounds, compositions, kits and unit dosage forms thereof, as well as processes for their preparation.

### TECHNOLOGICAL FIELD

The present disclosure provides self-emulsifying drug delivery systems for delivery of lipophilic compounds, as well as processes for their preparation.

### BACKGROUND ART

References considered to be relevant as background to the presently disclosed subject matter are listed below:
[1] Robinson, B T Gattefosse 1996, 89, 11-3
[2] Amidon et al., Pharm Res 1995, 12, 413-420
[3] Wadke et al., Pharmaceutical Dosage Forms: Tablets, 1. New York: Marcel Dekker; 1989. p. 1-73
[4] Serajuddin, J Pharm Sci 1999, 88, 1058-1066
[5] Aungst, J Pharm Sci 1993, 82, 979-986
[6] Toguchi et al., Chem Pharm Bull 1990, 38, 2792-2796
[7] Palin et al., Int J Pharm 1986, 33, 99-104
[8] Schwendener et al., J Cancer Res Clin Oncol 1996, 122, 723-726
[9] Dokania et al., Drug Delivery 2015, 22(6), 675-690
[10] Amri et al., Eur J Pharm Biopharm 2013, doi: 10.1016/j.ejpb.2013.10.015
[11] Yao et al, Biomed. Prev Nutr 2010, 1(1), 36-42
[12] Pund et al., Phytomedicine 2013, doi: 10.1016/j.phymed.2013.09.013.
[13] Pund et al., Colloids Surf B Biointerfaces 2014, 115, 29-36
[14] Iosio et al., Eur J Pharm Biopharm 2008, 69(2), 686-689
[15] Date et al., Nanomedicine 2010, 5, 1595-1616
[16] Obitte et al., J Pharm 2014, http://dx.doi.org/10.1155/2014/340486
[17] Patel et al., J Adv Pharm Technol Res. 2011, 2, 9-16
[18] Yadav et al., Sci World J. 2014, 2014, 1-10
[19] Cherniakov et al, Eur J Pharm Sci. 2017, 109, 21-30
[20] Shah et al., Int J Pharm. 1994, 106, 15-23
[21] Millar et al., Front. Pharmacol 2018, https://doi.org/10.3389/fphar.2018.01365
[22] Mechoulam et al., J. Clin. Pharmacol. 2002, 42, 11S-19S
[23] Welty et al., Epilepsy Currents 2014, 5, 250-252
[24] Huestis, Chem. Biodivers. 2007, 4, 1770-1804
[25] Zgair et al., Am. J. Transl. Res. 2016, 8, 3448-3459
[26] US 2018/0071210
[27] US 2017/0312244
[28] WO 2018/061007

Acknowledgement of the above references herein is not to be inferred as meaning that these are in any way relevant to the patentability of the presently disclosed subject matter.

### BACKGROUND

Medicinal herbal cannabis has been used for years in treating various therapeutic indications as well as alleviating pain and inflammatory-related syndromes. These treatments are based mainly on a specific group of lipophilic compounds, i.e. cannabinoids, found mainly in the resin-producing pistillate inflorescences of the cannabis plant, and although a variety of cannabinoid compounds have been identified over the years, two compounds are of particular interest for medicinal uses: tetrahydrocannabinol (THC) and cannabidiol (CBD). Despite the rapid and significant increase in the use of medicinal cannabis and the large number of clinical trials performed for various therapeutic indications, herbal cannabis and respective cannabinoids oil extracts have not met the rigorous regulatory requirements for medical approval, although approval has been obtained for specific well-characterized synthetic cannabinoids.

While the oral route has been the major administration route of various active compounds and drugs, oral delivery of 50% of the drug compounds is hindered due to the high lipophilicity of the administered drugs, including THC and CBD. Nearly 40% of new drug candidates exhibit low solubility in water, which leads to poor oral bioavailability, high intra- and inter-subject variability and lack of dose proportionality [1]. Thus, for such compounds, the absorption rate from the gastrointestinal (GI) lumen is controlled by dissolution [2].

Modification of the physicochemical properties, such as salt formation and particle size reduction of the compound may be one approach to improve the dissolution rate of the drug [3]. However, these methods have various limitations. For instance, salt formation of neutral compounds is not feasible and the synthesis of weak acid and weak base salts may not always be commercially practical. Moreover, salts that are formed may convert back to their original acid or base forms and lead to aggregation in the gastrointestinal tract. Particle size reduction may not be desirable in situations where handling difficulties and poor wettability are experienced for very fine powders [4]. To overcome these drawbacks, various other formulation strategies have been adopted including the use of cyclodextrins, nanoparticles, solid dispersions and permeation enhancers [5]. However, these delivery systems often cannot bypass the hepatic first-pass effect, which is one of main causes for the reduced oral bioavailability of cannabinoids.

In recent years, lipid-based formulations have attracted attention as a possible route to improve the oral bioavailability of poorly water-soluble drug compounds, especially in cases where such drugs are in the form of oils. It was shown that the incorporation of active lipophilic components into inert lipid vehicles, such as oils, surfactant-based dispersions, self-emulsifying formulations, emulsions and liposomes [3-8] may improve the oral bioavailability. However, it was also found that patient compliance for such formulations is relatively low due to their liquid form and the difficulty to mask their bitter taste.

Another approach is the incorporation of drug compounds into lipid formulations, such as lipid-based micro- and nano-emulsions, with a particular emphasis on Micro- or Nano-Self-Emulsifying Drug Delivery System (SMEDDS or SNEDDS, respectively) [9-13]. Self-emulsifying systems (SEDDS) are able to emulsify rapidly and spontaneously in the gastrointestinal fluids and create fine oil/water emulsions under the gentle agitation conditions provided by gastro-intestinal motion. The small droplets of oil increase drug diffusion into intestinal fluids (because of large surface area), along with faster and more uniform distribution of drug in the GI tract. They may also minimize the mucosal irritation due to the contact between the drug and the gut wall [14]. It was also reported that the mechanisms of oral bioavailability enhancement encompass improved solubility, changing intestinal permeability, and interfering with enzymes and transporter activity via bioactive lipid excipients and surfactants. Furthermore, bypassing hepatic first-pass metabolism was also reported as a result of oral lymphatic targeting of drugs [15].

General Description

To date, cannabinoids are typically orally administered in the form of an oil extract or alcoholic extracts that contain a variety of concentrations of cannabinoids. The content of cannabinoids in such oils is typically non-uniform from batch to batch, and is highly dependent on the type and quality of the herbal source and the extraction process used to obtain the oil. Such extracts elicit poor oral bioavailability and require frequent doses per day, reducing patients' compliance. Lipophilic compounds, cannabinoids being a mere example thereof, are known to be difficult to formulate, and most frequently are solubilized in oil solutions, packed in bottles and administered as metered volumes to be swallowed. These are also difficult to formulate into soft gelatin capsules due to their relatively low viscosity.

Self-emulsifying drug delivery systems (SEDDS) have been suggested to improve the therapeutic application of various aqueous poorly-soluble (i.e. lipophilic) drug molecules, by improving biopharmaceutical properties of the lipophilic compound [16-19]. In spite of some research done in this field, developing the proper combinations of components for a specific drug is typically a tedious and complex task. Despite the apparent simplicity of the formulation containing only few components, the obstacles are significant and many hurdles and challenges are encountered during the design of appropriate systems. Although SEDDS have several advantages, there are many limitations such as drug precipitation in vivo on dilution (especially following high dilution in physiologic fluids), encapsulation in soft or sealed hard gelatin capsules which are associated with few drawbacks such as manufacturing cost, and volatile solvent migration into the shells of soft or hard gelatin capsules resulting in the precipitation of the lipophilic drugs in the capsules, as well as leakage. Other drawbacks are the lack of good predictive in vitro models for the assessment of oxidation and polymorphism of the lipids used in formulating SEDDS. The need of efficient combination of components with the drug is key for the development of a successful SEDDS and represent a marked innovation in the design of the delivery system for oral administration of lipophilic compounds.

This disclosure provides self-emulsifying drug delivery systems (SEDDS) for oral delivery of lipophilic compounds and drugs, cannabinoids such as CBD and THC being an example, in controlled ratios and compositions, with improved oral bioavailability as well as increased patient compliance. The self-emulsifying system of this disclosure may also be used to modify the pharmacokinetic profile of the lipophilic drug, leading to reproducible enhanced delivery following oral administration resulting in a diminution of the dose and the reduction of adverse-effects without altering the efficacy of the drug.

Thus, in one of its aspects, the disclosure provides a self-emulsifying formulation for oral delivery of at least one lipophilic compound, the formulation comprising at least one lipophilic compound, at least one oil in a content of at least 10 wt % of the formulation, at least one surfactant, and at least one structurant.

The term self-emulsifying formulation (or self-emulsifying drug delivery systems, SEDDS) refers to an isotropic and thermodynamically stable oily solution that can be used as a pre-concentrate. This formulation (or pre-concentrate) is a mixture comprising oil, surfactants, and structurants (e.g. co-solvents/co-surfactants), capable of solubilizing or dissolving lipophilic compounds. Upon introduction into an aqueous liquid, the formulation emulsifies (i.e. forms an emulsion) spontaneously under mild agitation. In vivo motility of the stomach and intestine, for example, provides sufficient agitation required for self-emulsification [20]. Thus, such systems are typically essentially or completely free of water, and are administered as such or mixed into an aqueous diluent shortly prior to administration. Thus, in some embodiments, the self-emulsifying formulation is essentially devoid of water. The expression essentially water free (or essentially devoid of water) means to denote formulations that contain up to 5 wt % of water. In other embodiments, the formulation is free of water.

The lipophilic compound is a compound, e.g. a drug or a nutritional supplement that is poorly dissolved in water, and is typically solubilized in oil or oily components. In some embodiments, the lipophilic compound is any compound or therapeutic active ingredient that has a (log P) >2 in octanol/water. The self-emulsifying formulation of this disclosure is tailored to stabilize and solubilize a variety of lipophilic compounds, for example, cannabinoids. Another example of lipophilic compounds are CB1 receptor blockers which exhibit a lipophilic nature with a log P>2, and a molecular weight ranging from 150 to 1200 Da (for example those described in PCT application no. PCT/IL2020/050062, the content of relevant parts of which is incorporated herein by reference).

In some embodiments, the lipophilic compound may be selected from cannabinoids, CB1 receptor blockers with molecular weights ranging from 150 to 1200 Da, oxaliplatin palmitate acetate (OPA), cyclosporine A, a vitamin, an anti-oxidant, a lipid, a hormone, an antibiotic agent, a prophylactic agent, a small molecule of a molecular weight of less than about 1,000 Da or less than about 500 D, an analgesic or anti-inflammatory agent; an anthelmintic agent; an anti-arrhythmic agent; an anti-bacterial agent; an anti-coagulant; an anti-depressant; an antidiabetic; an anti-epileptic; an anti-fungal agent; an anti-gout agent; an anti-hypertensive agent; an anti-malarial agent; an anti-migraine agent; an anti-, muscarinic agent; an anti-neuroplastic agent or immunosuppressant; an anti-protazoal agent; an anti-thyroid agent; an alixiolytic, sedative, hypnotic or neuroleptic agent; a beta-blocker; a cardiac inotropic agent; a corticosteroid; a diuretic agent; an anti-Parkinsonian agent; a gastro-intestinal agent; an histamine H1-receptor antagonist; a lipid regulating agent; a nitrate or anti-anginal agent; a nutritional agent; an HIV protease inhibitor; an opioid analgesic; capsaicin a sex hormone; a cytotoxic agent; and a stimulant agent, and any combination of the aforementioned.

In some embodiments, the lipophilic compound may be selected from cannabinoids, e.g. CBD, THC or mixtures thereof.

In other embodiments, the lipophilic compound is at least one CB1 receptor blocker with molecular weights ranging from 150 to 1200 Da. Exemplary CBD1 receptor blockers can be selected from:

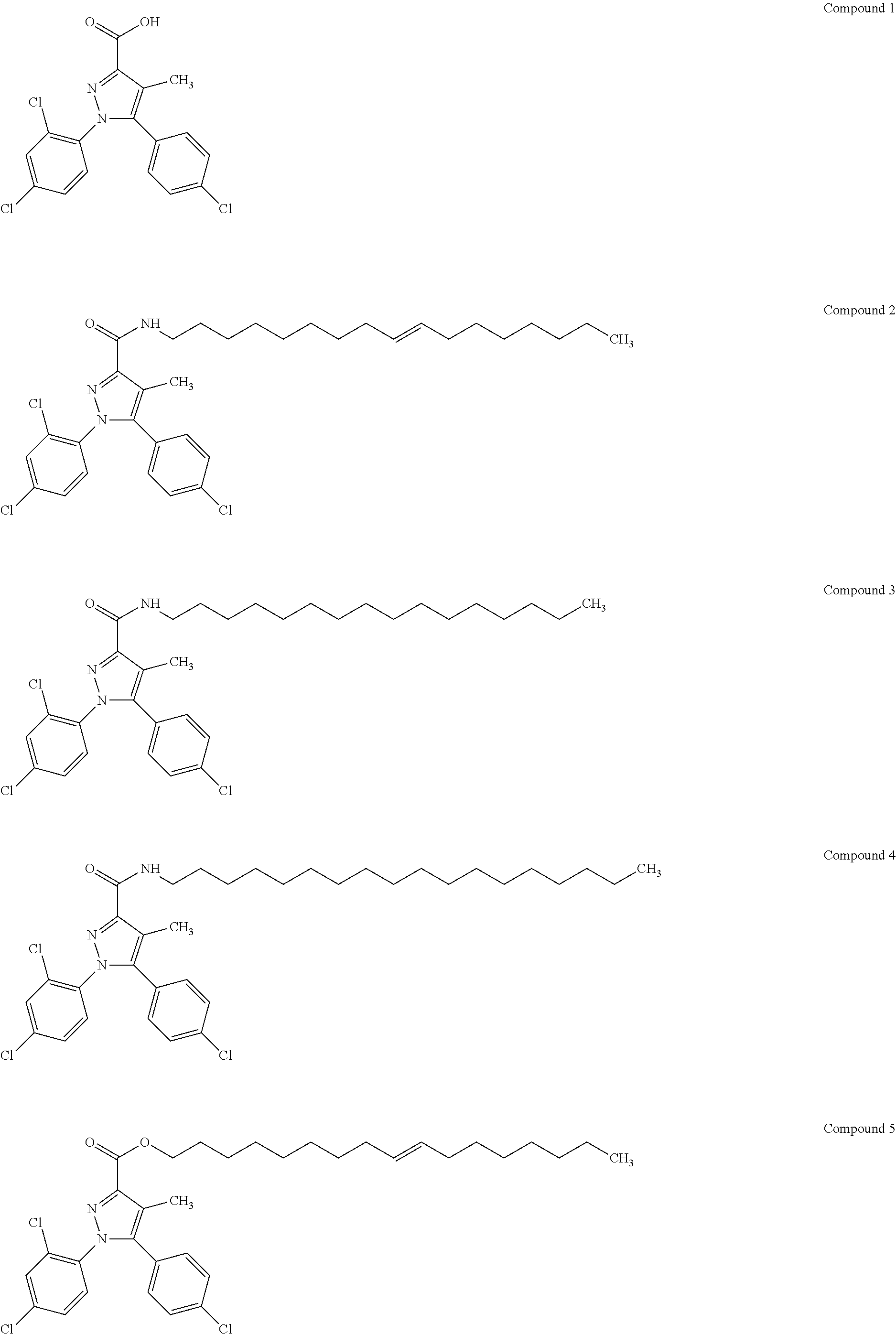 [SMILES: Cc1c(C(=O)O)nn(-c2ccc(Cl)cc2Cl)c1-c1ccc(Cl)cc1 ; CCCCCCC/C=C/CCCCCCCCNC(=O)c1nn(-c2ccc(Cl)cc2Cl)c(-c2ccc(Cl)cc2)c1C ; CCCCCCCCCCCCCCCCNC(=O)c1nn(-c2ccc(Cl)cc2Cl)c(-c2ccc(Cl)cc2)c1C ; CCCCCCCCCCCCCCCCCCNC(=O)c1nn(-c2ccc(Cl)cc2Cl)c(-c2ccc(Cl)cc2)c1C ; CCCCCCC/C=C/CCCCCCCCOC(=O)c1nn(-c2ccc(Cl)cc2Cl)c(-c2ccc(Cl)cc2)c1C]

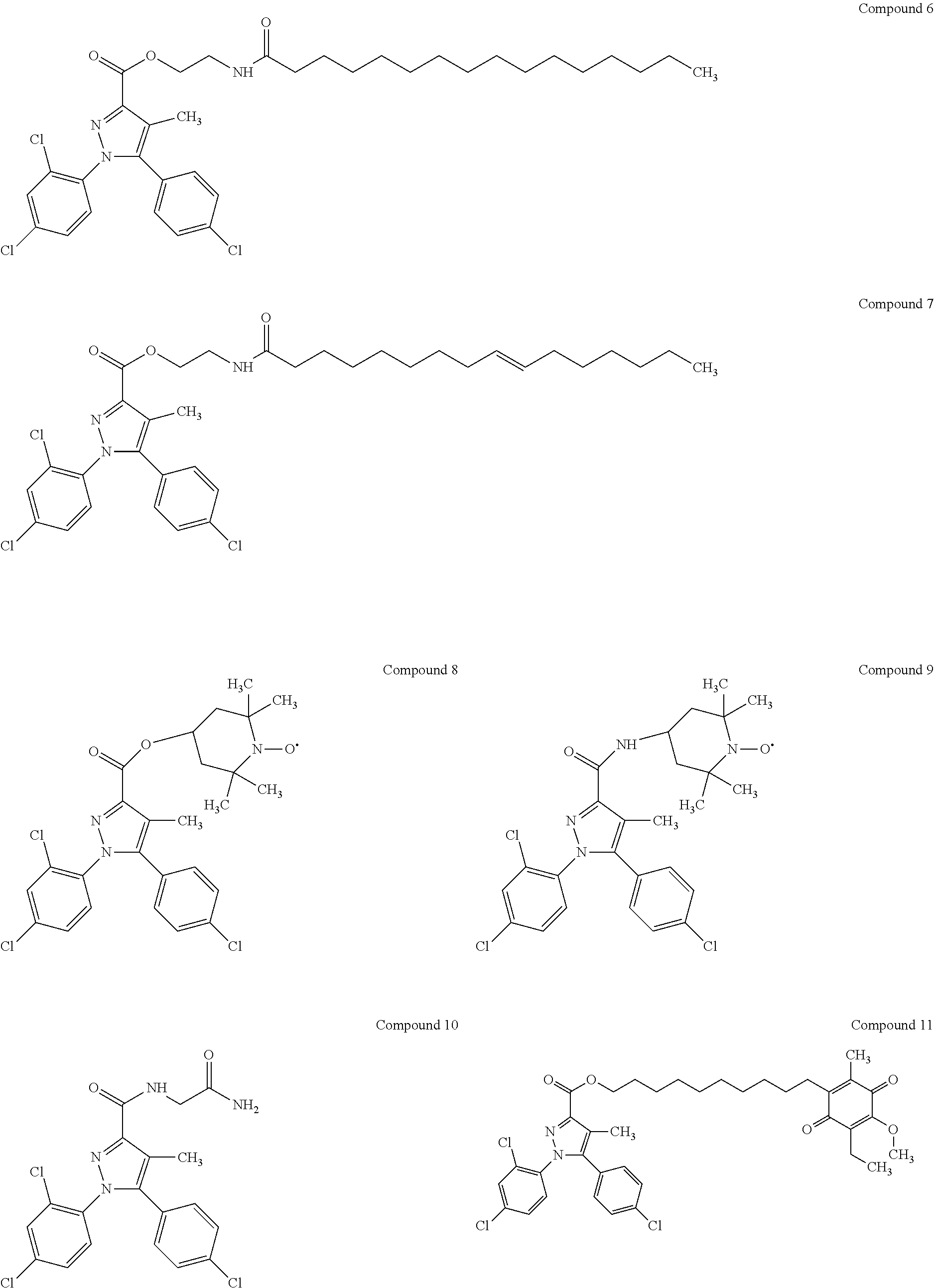

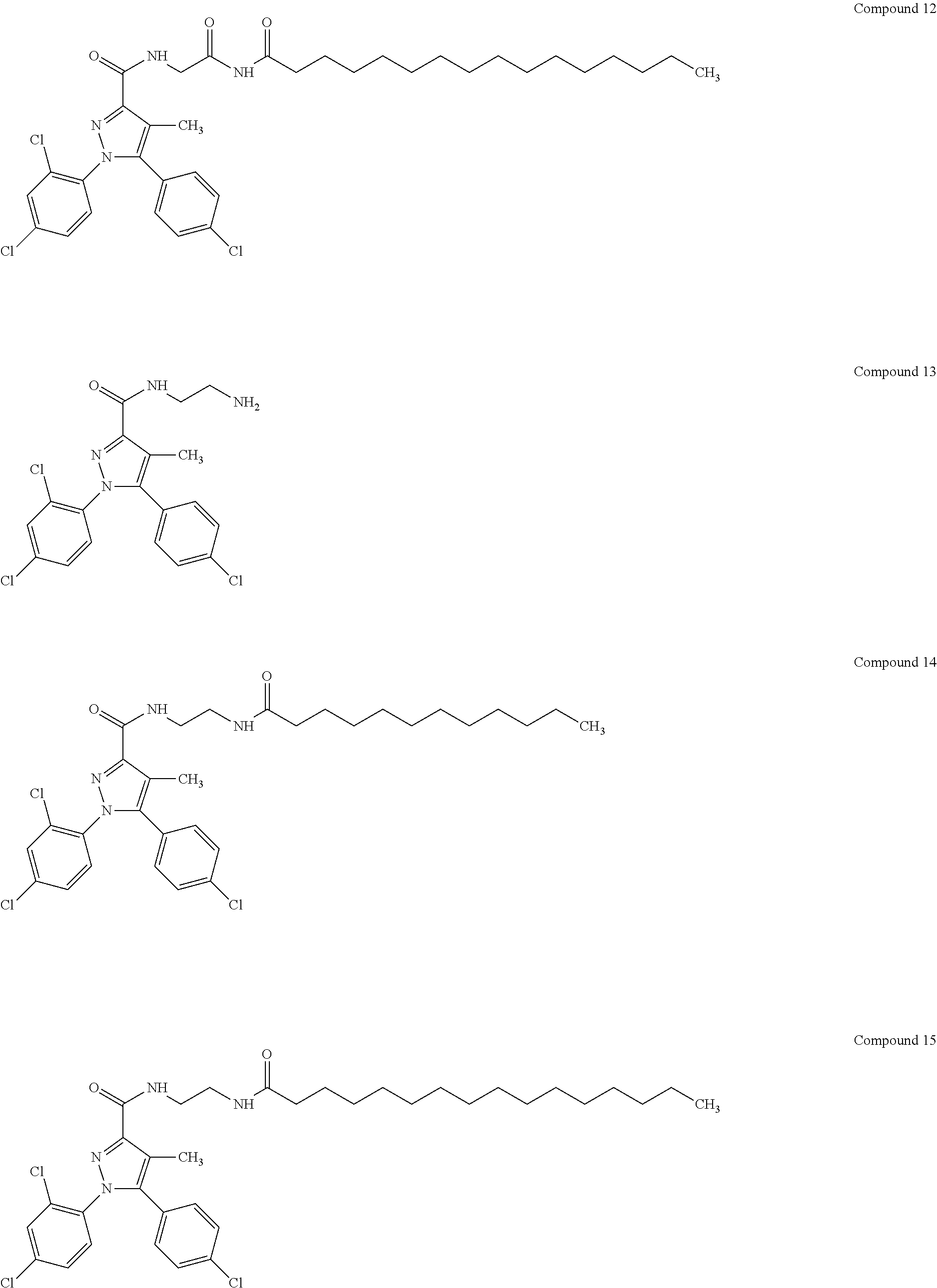 [SMILES: CCCCCCCCCCCCCCCC(=O)NC(=O)CNC(=O)c1nn(-c2ccc(Cl)cc2Cl)c(-c2ccc(Cl)cc2)c1C ; Cc1c(C(=O)NCCN)nn(-c2ccc(Cl)cc2Cl)c1-c1ccc(Cl)cc1 ; CCCCCCCCCCCC(=O)NCCNC(=O)c1nn(-c2ccc(Cl)cc2Cl)c(-c2ccc(Cl)cc2)c1C ; CCCCCCCCCCCCCCCC(=O)NCCNC(=O)c1nn(-c2ccc(Cl)cc2Cl)c(-c2ccc(Cl)cc2)c1C]

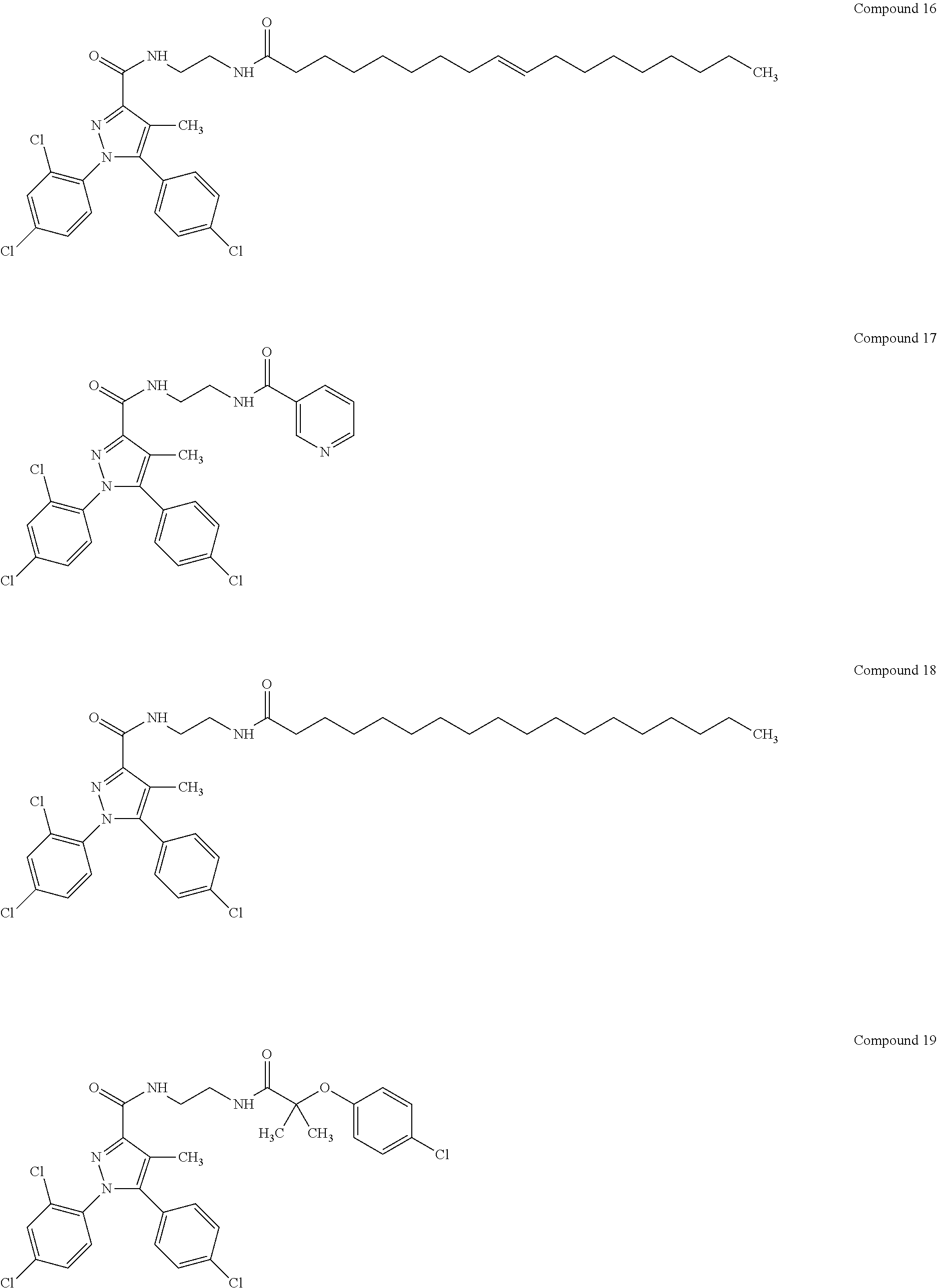 [SMILES: CCCCCCCC/C=C/CCCCCCCC(=O)NCCNC(=O)c1nn(-c2ccc(Cl)cc2Cl)c(-c2ccc(Cl)cc2)c1C ; Cc1c(C(=O)NCCNC(=O)c2cccnc2)nn(-c2ccc(Cl)cc2Cl)c1-c1ccc(Cl)cc1 ; CCCCCCCCCCCCCCCCCC(=O)NCCNC(=O)c1nn(-c2ccc(Cl)cc2Cl)c(-c2ccc(Cl)cc2)c1C ; Cc1c(C(=O)NCCNC(=O)C(C)(C)Oc2ccc(Cl)cc2)nn(-c2ccc(Cl)cc2Cl)c1-c1ccc(Cl)cc1]

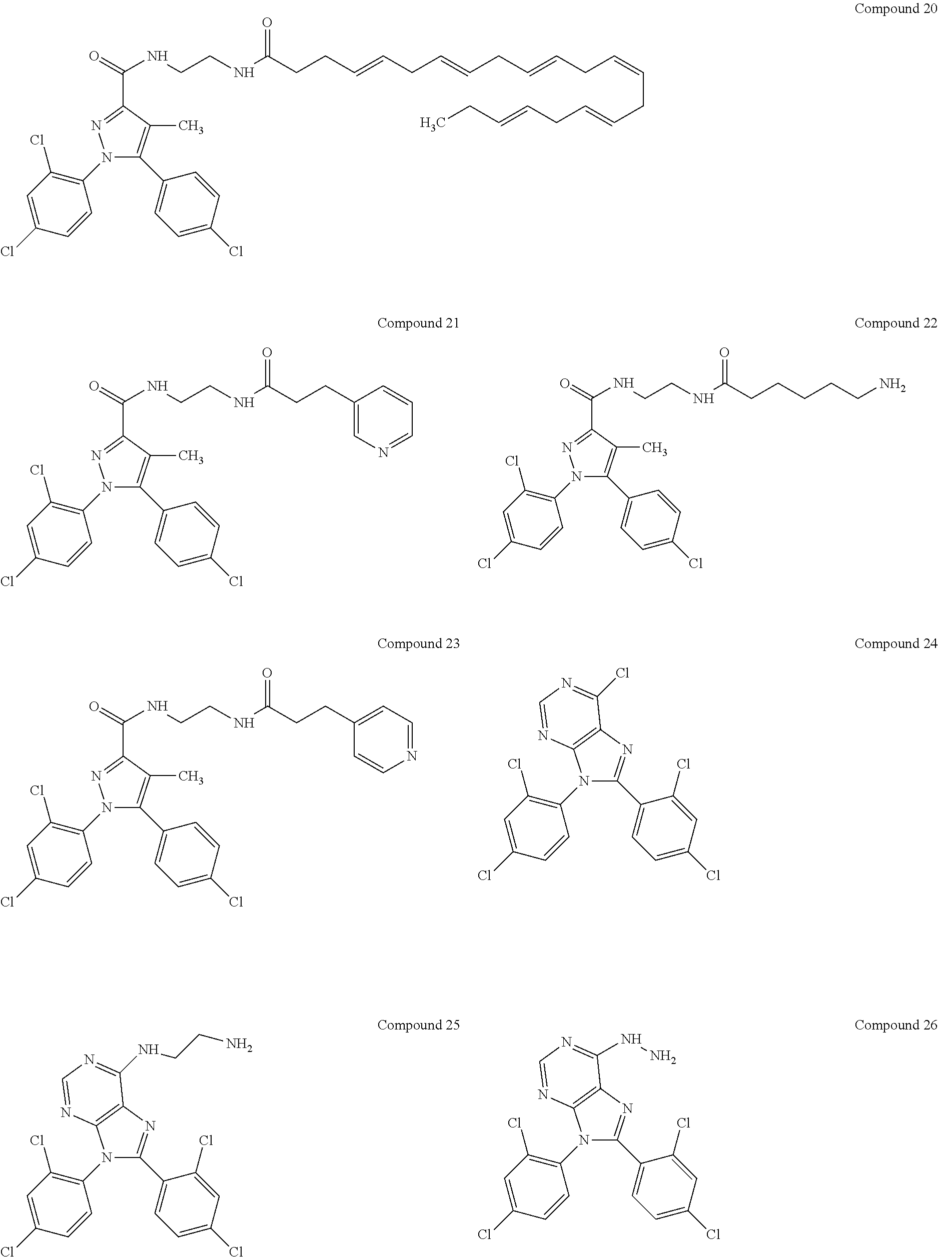

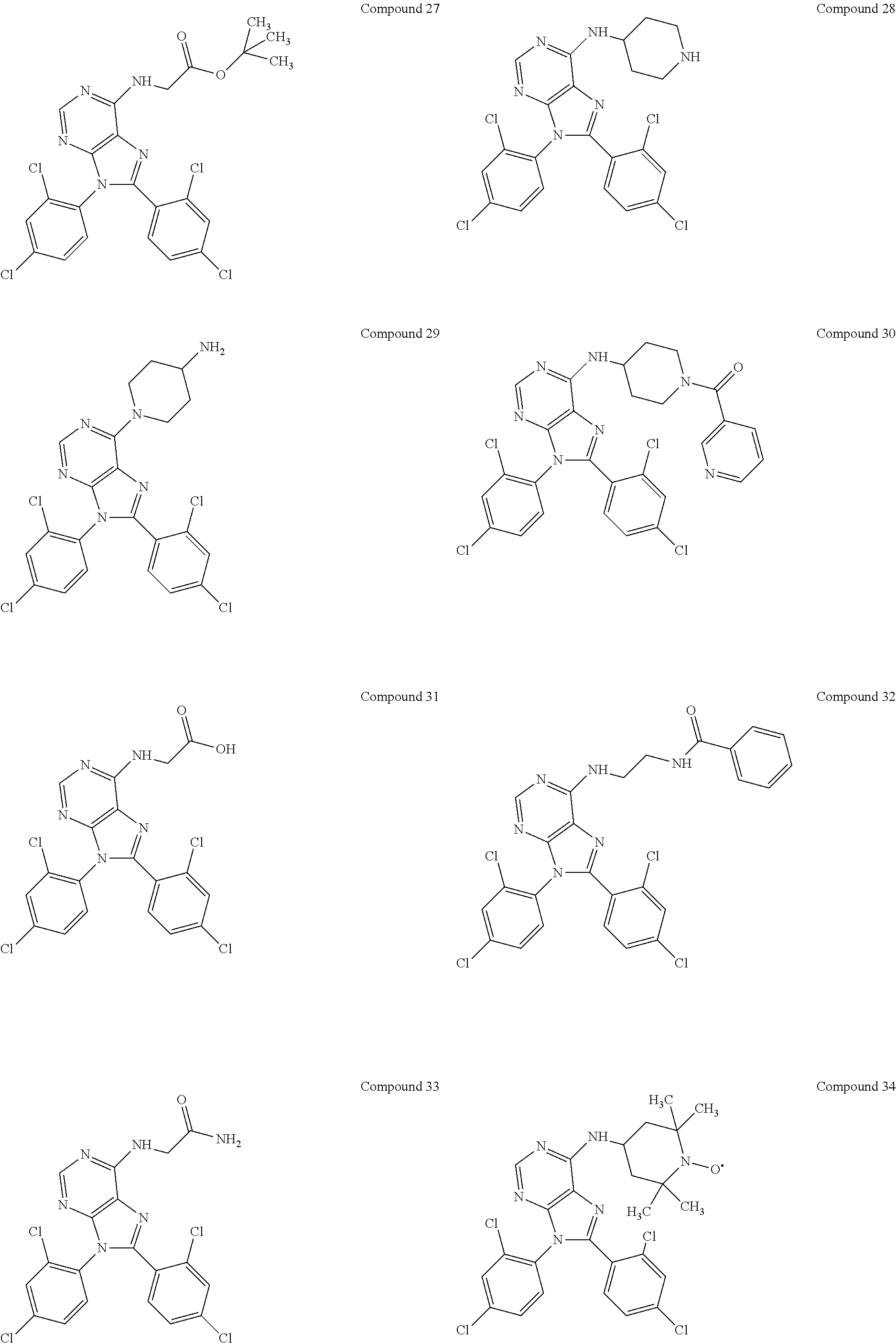

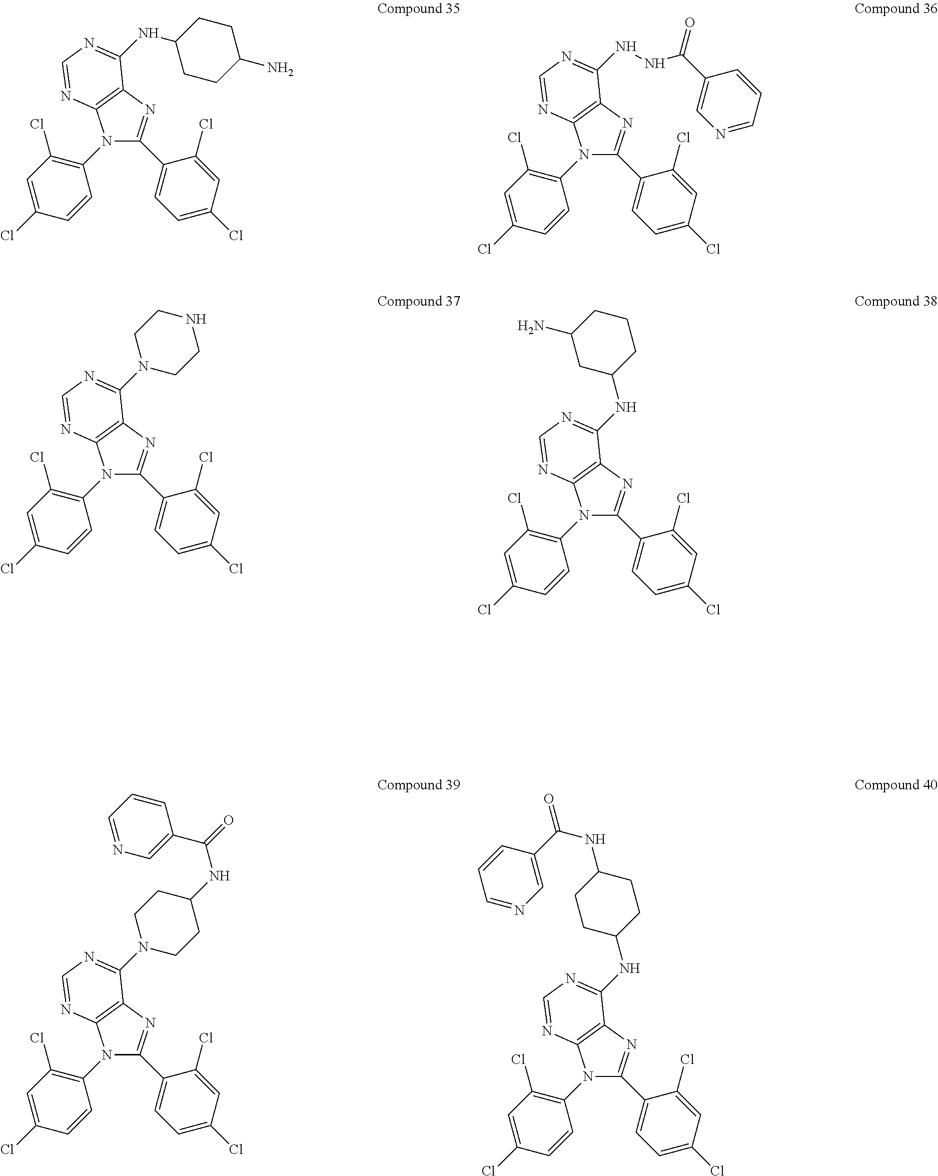 [SMILES: NC1CCC(Nc2ncnc3c2nc(-c2ccc(Cl)cc2Cl)n3-c2ccc(Cl)cc2Cl)CC1 ; O=C(NNc1ncnc2c1nc(-c1ccc(Cl)cc1Cl)n2-c1ccc(Cl)cc1Cl)c1cccnc1 ; Clc1ccc(-c2nc3c(N4CCNCC4)ncnc3n2-c2ccc(Cl)cc2Cl)c(Cl)c1 ; NC1CCCC(Nc2ncnc3c2nc(-c2ccc(Cl)cc2Cl)n3-c2ccc(Cl)cc2Cl)C1 ; O=C(NC1CCN(c2ncnc3c2nc(-c2ccc(Cl)cc2Cl)n3-c2ccc(Cl)cc2Cl)CC1)c1cccnc1 ; O=C(NC1CCC(Nc2ncnc3c2nc(-c2ccc(Cl)cc2Cl)n3-c2ccc(Cl)cc2Cl)CC1)c1cccnc1]

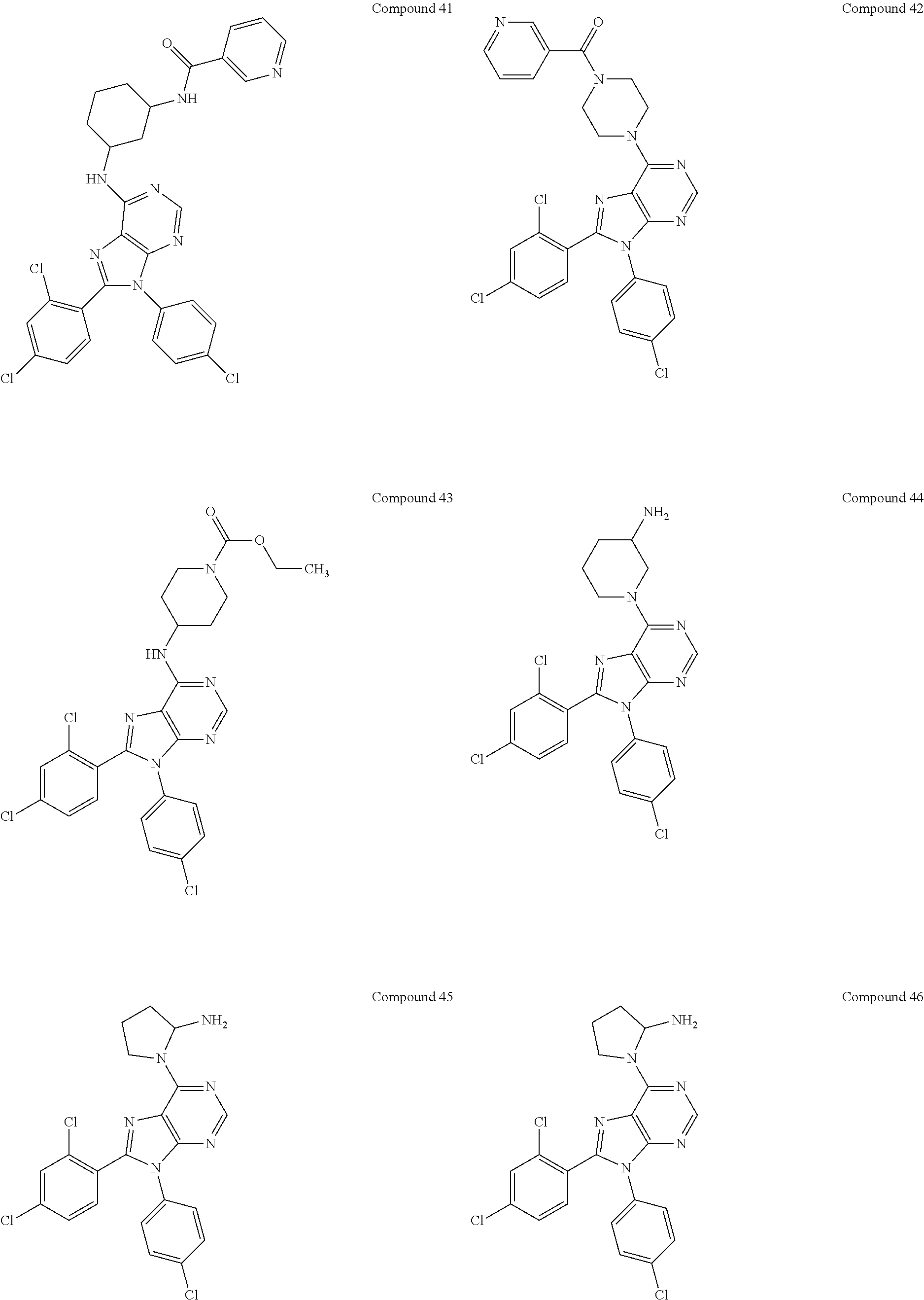 [SMILES: O=C(NC1CCCC(Nc2ncnc3c2nc(-c2ccc(Cl)cc2Cl)n3-c2ccc(Cl)cc2)C1)c1cccnc1 ; O=C(c1cccnc1)N1CCN(c2ncnc3c2nc(-c2ccc(Cl)cc2Cl)n3-c2ccc(Cl)cc2)CC1 ; CCOC(=O)N1CCC(Nc2ncnc3c2nc(-c2ccc(Cl)cc2Cl)n3-c2ccc(Cl)cc2)CC1 ; NC1CCCN(c2ncnc3c2nc(-c2ccc(Cl)cc2Cl)n3-c2ccc(Cl)cc2)C1 ; NC1CCCN1c1ncnc2c1nc(-c1ccc(Cl)cc1Cl)n2-c1ccc(Cl)cc1 ; NC1CCCN1c1ncnc2c1nc(-c1ccc(Cl)cc1Cl)n2-c1ccc(Cl)cc1]

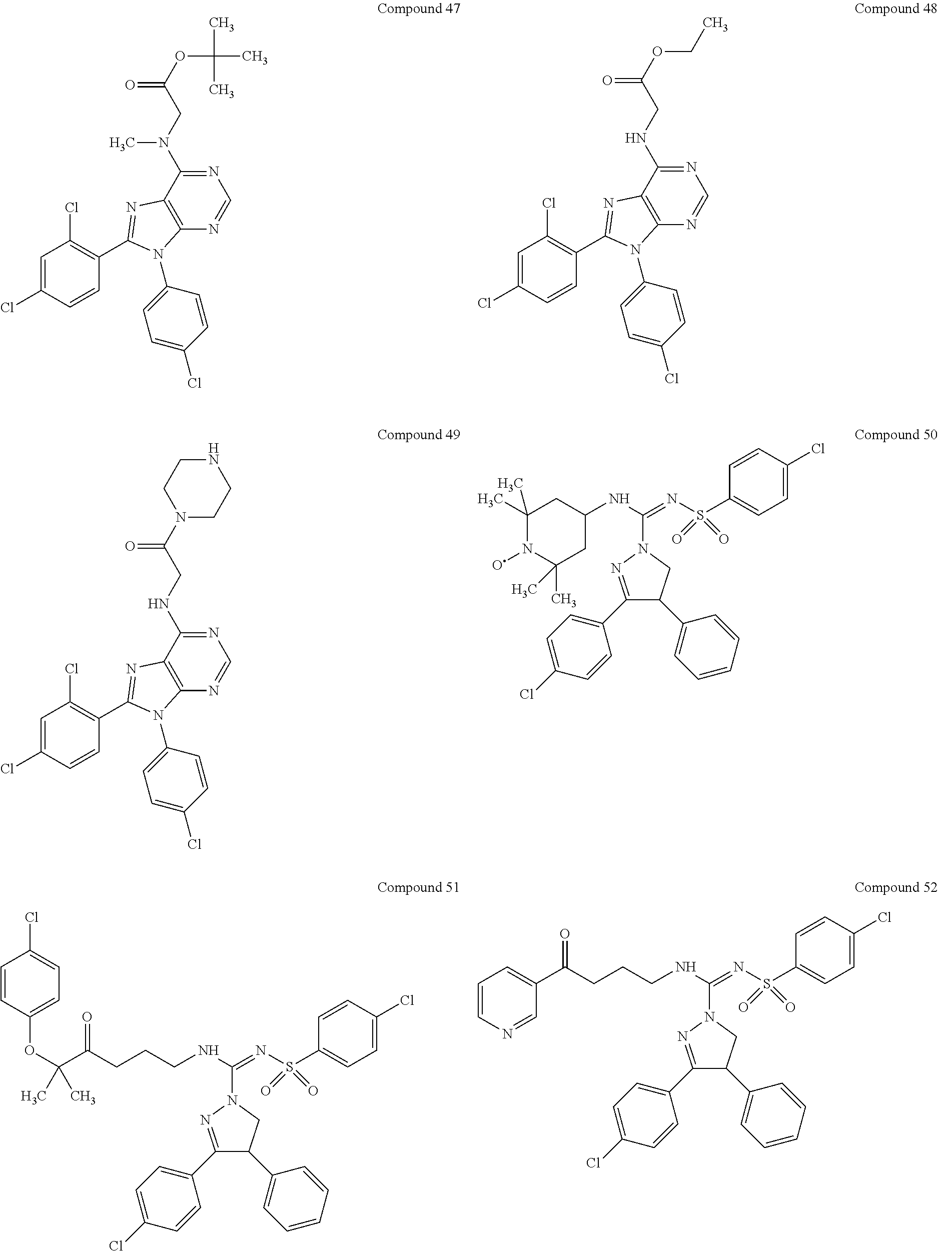

Cannabinoid-loaded self-emulsifying formulations (cannabinoid loaded SEDDS) are specific embodiments of this disclosure. The cannabinoid-loaded self-emulsifying formulation comprises at least one cannabinoid, at least one oil in a content of at least 10 wt % of the formulation, at least one surfactant, and at least one structurant.

In some embodiments, the formulation is adapted to spontaneously form a nanoemulsion when diluted with an aqueous diluent. In the context of the present disclosure the term nanoemulsion refers to emulsions having a droplet mean diameter of between about 100 nm and 800 nm, 100 nm and 500 nm, typically between about 100 nm and 300 nm that are formed when the formulation is diluted with said aqueous diluent.

The droplet mean diameter (or droplet size) refers to the arithmetic mean of measured droplets' diameters, wherein the diameters range ±15% from the mean value.

The spontaneous formation of emulsions or nanoemulsions when diluted in an aqueous diluent typically depends on the components of the formulation and their relative amounts.

In some embodiments, the components of the formulation are selected such that the formulation forms oily droplets having a droplet-diluent interface energy of greater than zero when diluted in said aqueous diluent. A non-zero interface energy causes the formulation to emulsify when introduced to water and maintain it in a kinetically stable state for a defined period of time.

Hence, in another aspect, there is provided a cannabinoid-loaded self-emulsifying formulation comprising at least one cannabinoid, at least one oil in a content of at least 10 wt % of the formulation, at least one surfactant, and at least one structurant, adapted to form oily droplets having a droplet-diluent interface energy of greater than zero when diluted in an aqueous diluent.

One of the main components determining the interfacial energy is the relatively high content of oil, when compared to other self-emulsifying formulations. In the formulations of this disclosure, the content of the oil is at least 10 wt % of the formulation, resulting in a relatively large droplet size (i.e. above 100 nm), as well as relatively efficient decomposition of the droplet after intake.

In the context of the present disclosure, the term oil refers to natural or synthetic oil in which the lipophilic compound is dissolved. The oil may be selected from mineral oil, paraffinic oils, vegetable oils, glycerides, esters of fatty acids, liquid hydrocarbons and others, as well as mixtures thereof.

According to some embodiments, the oil may be selected from tripropionin, tributyrin, hydrogenated vegetable oils, nut oils, anise oil, soybean oil, hydrogenated soybean oil, apricot kernel oil, corn oil, olive oil, peanut oil, almond oil, walnut oil, cashew oil, rice bran oil, poppy seed oil, cottonseed oil, canola oil, sesame oil, hydrogenated sesame oil, coconut oil, flaxseed oil, cinnamon oil, clove oil, nutmeg oil, coriander oil, lemon oil, orange oil, safflower oil, cocoa butter, palm oil, palm kernel oil, sunflower oil, rapeseed oil, castor oil, hydrogenated castor oil, polyoxyethylene oil derivatives, mid-chain triglycerides (MCT), glyceryl monooleate (Type 40) [Peceol™, Gattefosse], and mixtures thereof.

According to other embodiments, the oil is tripropionin.

According to another embodiment, the oil is tributyrin.

According to a further embodiment, the oil is selected from tripropionin, tributyrin and mixtures thereof.

The oil may be present in the formulation, according to some embodiments, at an amount of between about 10 and 60 wt %. In other embodiments, the oil may be present in the formulation in an amount between 10 and 50 wt %, between 10 and 45 wt %, between 10 and 40 wt %, between 10 and 35 wt %, or even between 10 and 30 wt %. In some other embodiments, the oil may be present in the formulation in an amount between 15 and 50 wt %, between 15 and 45 wt %, between 15 and 40 wt %, between 15 and 35 wt %, or even between 15 and 30 wt %.

According to further embodiments, the oil comprises at least one first oil and at least one second oil. According to such embodiments, said at least one first oil may be selected from tripropionin, tributyrin or a combination thereof, and said at least one second oil may be selected from hydrogenated vegetable oils, nut oils, anise oil, soybean oil, hydrogenated soybean oil, apricot kernel oil, corn oil, olive oil, peanut oil, almond oil, walnut oil, cashew oil, rice bran oil, poppy seed oil, cottonseed oil, canola oil, sesame oil, hydrogenated sesame oil, coconut oil, flaxseed oil, cinnamon oil, clove oil, nutmeg oil, coriander oil, lemon oil, orange oil, safflower oil, cocoa butter, palm oil, palm kernel oil, sunflower oil, rapeseed oil, castor oil, hydrogenated castor oil, polyoxyethylene oil derivatives, mid-chain triglycerides (MCT), glyceryl monooleate (Type 40), and mixtures thereof. The total amount of said at least one first oil and at lease one second oil in the formulation is at least 10 wt %.

The formulation comprises at least one surfactant. The term surfactant refers to ionic or non-ionic surfactants, which may have a hydrophilic nature, i.e. a surfactant having an affinity for water. In some embodiments, the at least one surfactant is selected from polyoxyl castor oil (e.g. Cremophor RH40, Kolliphor RH40), polysorbate 80, oleoyl polyoxyl-6 glycerides (Labrafil M1944 CS), polyoxyl 35 hydrogenated castor oil, sucrose distearate, tocopherol polyethylene glycol 1000 succinate (TPGS), lauroyl polyoxyl-32 glycerides (Gelucire), sorbitan monooleate, low-HLB polyoxylglycerides (Labrafil® M 1944 CS and Labrafil® M 2125 CS), linoleoyl polyoxy-6-glycerides, and combinations thereof.

The formulation may comprise, by some embodiments, between about 10% and 50 wt % of said at least one surfactant.

The formulation further comprises at least one structurant. The term structurant should be understood to encompass any agent which is capable (together with the surfactant) of modifying the interfacial tension/energy between the oil phase and an aqueous phase, allowing for the spontaneous formation of an emulsion or a nanoemulsion once the formulation is mixed with an aqueous diluent. The term is meant to encompass co-surfactants and co-solvents, as well as components which can function both a co-surfactant and a co-solvent. The combination of surfactants and structurants in the formulation described herein renders the formulations with a droplet-diluent interface energy of greater than zero once diluted in an aqueous diluent. The structurant may be selected from one or more polyols, diglycerides, polyoxyethylenes, and others.

According to some embodiments, the at least one structurant can be selected from polyethylene glycol (PEG), propylene glycol (PG), glycerin, and combinations thereof.

The at least one structurant may, by some embodiments, be present in the formulation in a content of at least 10 wt %. According to other embodiments, the at least one structurant is present in the formulation in an amount of between about 10 and 50 wt %.

In some embodiments, said at least one structurant may have an average molar mass of up to about 600 g/mol. In some other embodiments, said at least one structurant may have an average molar mass of above about 600 g/mol.

According to some embodiments, the formulations may comprise at least two structurants, present in the formulation in a total content of at least 10 wt %, e.g. between about 10 and 50 wt % of the formulation.

In some embodiments, the formulations of this disclosure comprise a first structurant having an average molecular mass of up to about 600 g/mol, and a second structurant having an average mass equal to or greater than about 800 g/mol.

The formulations of this disclosure may be provided in a liquid form or semi-solid form, depending, inter alia, on the type of structurants used in the formulation. The term semi-solid refers to a formulation having a viscosity of at least 10 cps (centipois) at 25° C., for example between about 10 and 10,000 cps at 25° C.

As noted above, the self-emulsifying formulations of this disclosure can be used as a delivery system for cannabinoids and may be tailored to solubilize various cannabinoids.

Cannabinoids are a group of psychoactive and non-psychoactive compounds which have an activity on cannabinoid receptors in cells to repress neurotransmitter release in the brain. The term is meant to encompass cannabinoids which are obtained from natural sources by various processes of treatment or extraction, as well as to synthetically obtained cannabinoids. The cannabinoid may be selected from one or more of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidiorcol (CBD-C₁), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C₄ (THCA-C₄), delta-9-tetrahydrocannabinol-C₄ (THCA-C₄), delta-9-tetrahydrocannabivarinic acid (THCVA), delta-9-tetrahydrocannabivarin (THCV), delta-9-tetrahydrocannabiorcolic acid (THCA-C₁), delta-9-tetrahydrocannabiorcol (THC-C₁), delta-7-cis-iso-tetrahydrocannabivarin, delta-8-tetrahydrocannabinolic acid A (Δ⁸-THCA), delta-8-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C₄ (CBN-C₄), cannabivarin (CBV), cannabinol-C₂ (CBN-C₂), cannabiorcol (CBN-C₁), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahtdro-7-hydroxy-α-α-2-trimethyl-9-n-propyl-2,6-methano-2H-1-benzoxocin-5-methanol (OH-iso-HHCV), cannabiripsol (CBR), trihydroxy-delta-9-tetrahydroxycannabinol (triOH-THC), and any other cannabinoid.

By some embodiments, the formulations disclosed herein may comprise at least 0.05 wt % of said at least one cannabinoid. According to other embodiments, the formulations may comprise between about 0.05 and 40 wt % of said at least one cannabinoid, e.g. between about 1 and 40 wt % of said at least one cannabinoid.

In some embodiment, the cannabinoid is CBD. The formulations may, by some embodiments, comprise at least 0.05 wt % of CBD. By other embodiments, the formulations may comprise between about 0.05 wt % and 15 wt % of CBD, or even between about 1 and 15 wt % CBD.

In other embodiments, the cannabinoid is THC. The formulations may, by some embodiments, comprise at least 0.05 wt % of THC. By other embodiments, the formulations may comprise between about 0.1 wt % and 10 wt % of THC.

In some embodiments, the formulations comprise both CBD and THC. In such embodiments, the weight ratio of CBD to THC in the formulation may ranges between 20:1 and 1:20. In some embodiments, the weight ratio of CBD to THC in the formulation may be 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1 or 1:1. In other embodiments, or the weight ratio of CBD to THC in the formulation may be 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2 or 1:1.

A self-emulsifying composition for solubilization of at least one lipophilic compound, i.e. a composition into which the lipophilic compound can be solubilized, is also an aspect of this disclosure. The self-emulsifying composition comprises at least one oil in a content of at least 10 wt % of the composition, at least one surfactant, and at least one structurant. The self-emulsifying composition can be loaded with one or more lipophilic compounds in order to form the self-emulsifying formulations described herein. Each of the oils, surfactants and structurants of the self-emulsifying composition are as described herein in connection with the self-emulsifying formulation.

According to another aspect of this disclosure, there is provided a cannabinoid-loaded self-emulsifying formulation comprising at least one cannabinoid, at least one oil in a content of at least 10 wt % of the formulation, at least one surfactant, and at least one structurant, said at least one oil comprising tripropionin. Each of the surfactants and structurants of the self-emulsifying composition are as described herein in connection with the self-emulsifying formulation.

According to a further aspect of this disclosure, there is provided a cannabinoid-loaded self-emulsifying formulation comprising at least one cannabinoid, at least one oil in a content of at least 10 wt % of the formulation, at least one surfactant, and at least one structurant, said at least one oil comprising tributyrin. Each of the surfactants and structurants of the self-emulsifying composition are as described herein in connection with the self-emulsifying formulation.

According to yet another aspect of this disclosure, there is provided a cannabinoid-loaded self-emulsifying formulation comprising at least one cannabinoid, at least one oil in a content of at least 10 wt % of the formulation, at least one surfactant, and at least one structurant, said at least one oil comprising at least one first oil selected from tripropionin, tributyrin and mixtures thereof, and said at least one second oil comprises MCT. Each of the surfactants and structurants of the self-emulsifying composition are as described herein in connection with the self-emulsifying formulation.

In another aspect, there is provided a cannabinoid-loaded self-emulsifying formulation comprising at least one cannabinoid, at least one oil in a content of at least 10 wt % of the formulation, at least one surfactant, and at least one structurant, adapted to form oily droplets having a mean diameter of at least 100 nm when diluted with said aqueous diluent, the droplets being dispersed in a continuous phase constituted by the aqueous diluent. Each of the oils, surfactants and structurants of the self-emulsifying composition are as described herein in connection with the self-emulsifying formulation.

By another one of its aspects, this disclosure provides a process for preparing the self-emulsifying formulations described herein, the process comprising solubilizing at least one lipophilic compound (e.g. at least one cannabinoid) in a self-emulsifying composition that comprises at least 10 wt % oil, at least one surfactant, and at least one structurant to obtain a mixture, and homogenizing the mixture under suitable conditions to obtain a lipophilic compound-loaded self-emulsifying formulation. Each of the lipophilic compounds, oils, surfactants and structurants of the self-emulsifying composition are as described herein in connection with the self-emulsifying formulation.

In another aspect, there is provided a process for preparing the self-emulsifying formulations described herein, the process comprising the steps of:

(a) mixing at least 10 wt % oil, at least one surfactant, and at least one structurant to obtain a self-emulsifying composition; and

(b) solubilizing at least one lipophilic compound (e.g. at least one cannabinoid) in the self-emulsifying composition to obtain a lipophilic compound-loaded self-emulsifying formulation.

Each of the oils, surfactants and structurants of the self-emulsifying composition are as described herein in connection with the self-emulsifying formulation.

The solubilizing of step (b) may, by some embodiments, be carried out under suitable conditions (e.g. mixing and/or heating) to obtain a homogenous solution, thus obtaining the lipophilic compound-loaded self-emulsifying formulation.

Mixing may be carried out by any suitable known method, for example, manual mixing, magnetically stirring, mixing by pedals and others. In some embodiments, the mixing is carried out for between about 5 and 60 minutes. In other embodiments, the mixing is carried out at a temperature of between about 30 and 60° C.

As will become apparent from this disclosure, the formulations of this disclosure may be particularly suitable for oral delivery of various lipophilic compounds, for example various cannabinoids. As already noted, the self-emulsifying formulations of this disclosure can spontaneously emulsify into an emulsion or a nanoemulsion when mixed with gastric fluids, the formulations of this disclosure can be adapted for administration as such, i.e. without any pre-dilution before administration.

In some embodiments, the formulation may be adapted for oral delivery of said lipophilic drug (e.g. cannabinoid) as such. In other embodiments, the formulation may be administered in a diluted form, namely in the form of an emulsion or a nanoemulsion formed before administration by diluting the formulations of this disclosure with an aqueous diluent.

Thus, another aspect of this disclosure is an emulsion for oral delivery of at least one lipophilic compound (e.g. at least one cannabinoid), the emulsion comprising oily droplets of the formulation as described herein, dispersed in a continuous phase constituted by an aqueous diluent. The emulsion may be a nanoemulsion.

In some embodiments, the emulsion is a nanoemulsion, with a droplets' mean diameter of at least 100 nm, e.g. between about 100 nm and 800 nm, between about 100 nm and 500 nm, or even between about 100 and 300 nm.

In yet another aspect, this disclosure provides a process for obtaining an emulsion or nanoemulsion as described herein, the process comprising mixing a self-emulsifying formulation disclosed herein with at least one aqueous diluent to obtain spontaneously said emulsion.

This disclosure also provides, in another aspect, a pharmaceutical composition comprising the self-emulsifying formulations disclosed herein.

In some embodiments, the pharmaceutical composition may comprise at least one pharmaceutically acceptable carrier. The pharmaceutically acceptable carriers described herein, for example, vehicles, adjuvants, excipients, or diluents, are well-known to those who are skilled in the art and are readily available. It is preferred that the pharmaceutically acceptable carrier be one which is chemically inert to the active compounds and one which has no detrimental side effects or toxicity under the conditions of use. The choice of carrier will be determined in part by the lipophilic compound (i.e. cannabinoid), as well as by the particular method used to administer the composition. Accordingly, there is a wide variety of suitable carriers for the pharmaceutical composition of the present disclosure.

In some embodiments, the pharmaceutical composition further comprises an aqueous diluent. In the context of the present disclosure, the term aqueous diluent should be understood to refer to any liquid having water as a main component thereof. The aqueous diluent may be selected from water, saline, dextrose solution, water/alcohol mixtures, sweetener-containing aqueous solutions, flavor-containing aqueous solutions, an isotonic solution, etc.

The pharmaceutical composition may comprise a variety of additives, depending on the administration route and/or desired properties of the pharmaceutical composition, such as anti-oxidants, buffers, bacteriostats, suspending agents, solubilizers, thickening agents, gelling agent, stabilizers, preservatives, viscosity increasing agents, coloring agents, a fragrance, flavoring agents, flavor masking agents, absorbers, fillers, electrolytes, proteins, chelating agents, and others. However, it is to be noted that the additives should be selected such that the self-emulsifying properties of the formulations, as well as their pharmacokinetic properties are not hindered by such addition.

By another aspect, there is provided a unit dosage form for oral delivery of at least one lipophilic compound (e.g. at least one cannabinoid), the unit dosage form comprising the formulation disclosed herein.

In some embodiments, the unit dosage form may be in a form selected from a is in a form selected from a spray, a reconstitutable concentrate, an oil, a capsule, a soft-gel capsule, a gel, an emulsion, or a syrup. The unit dosage form may comprise the self-emulsifying formulation as such or may comprise an emulsion or nanoemulsion of the formulation (namely an emulsion formed by the formulation and a suitable aqueous diluent).

In another aspect of this disclosure there is provided a kit comprising a formulation as disclosed herein and an aqueous diluent.

The kit may comprise at least one first container holding the formulation and at least one second container holding the aqueous diluent. In some embodiments, each of the first container and the second container may, independently, comprise a plurality of compartments, each compartments containing a volume of formulation or aqueous diluent to prepare a single dose of emulsion.

In other embodiments, the kit may comprise a first container holding the formulation and a second container holding the aqueous diluent, the first and second container being integrally formed one with the other, and comprising a breakable seal therebetween. In such kits, a user can break the seal upon demand, thus causing the volumes of the first container and the second container to be fluidly linked, permitting mixing of the formulation into the diluent for forming the emulsion immediately prior to administration.

The kit may further comprise measuring means, e.g. a syringe, a graduated pipette, a measuring cup, a graduated mixing vessel, etc. to permit a user to measure a defined amount of formulation and aqueous diluent from the first and second containers, respectively, for preparing the emulsion prior to administration.

A further aspect, provides a method of treating a subject suffering from a condition or a disorder, the method comprising orally administering to the subject an effective amount of the formulations, emulsions or nanoemulsions, pharmaceutical compositions or unit dosage forms disclosed herein.

In another aspect there is provided formulations, emulsions or nanoemulsions, pharmaceutical compositions or unit dosage forms disclosed herein for use in treating a condition or disorder in a subject in need thereof.

In some embodiments, the condition or disorder may be selected from pain associated disorders (as an analgesic), inflammatory disorders and conditions (as anti-inflammatory), apatite suppression or stimulation (as anoretic or stimulant), symptoms of vomiting and nausea (as antiemetic), intestine and bowl disorders, disorders and conditions associated with anxiety (as anxiolytic), disorders and conditions associated with psychosis (as antipsychotic), disorders and conditions associated with seizures and/or convulsions (as antiepileptic or antispasmodic), sleep disorders and conditions (as anti-insomniac), disorders and conditions which require treatment by immunosuppression, disorders and conditions associated with elevated blood glucose levels (as antidiabetic), disorders and conditions associated with nerve system degradation (as neuroprotectant), inflammatory skin disorders and conditions (such as psoriasis), disorders and conditions associated with artery blockage (as anti-ischemic), disorders and conditions associated with bacterial infections, disorders and conditions associated with fungal infections, proliferative disorders and conditions, disorders and conditions associated with inhibited bone growth, post trauma disorders, and others.

The formulations described herein may be used as such to induce at least one effect, e.g. therapeutic effect, or may be associated with at least one cannabinoid, which is capable of inducing, enhancing, arresting or diminishing at least one effect, by way of treatment or prevention of unwanted conditions or diseases in a subject. The formulations, emulsions or nanoemulsions, pharmaceutical compositions or unit dosage forms disclosed herein may be selected to treat, prevent or ameliorate any pathology or condition. The term treatment or any lingual variation thereof, as used herein, refers to the administering of a therapeutic amount of the formulations, emulsions or nanoemulsions, pharmaceutical compositions or unit dosage forms disclosed herein, which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above.

As known, the effective amount for purposes herein may be determined by such considerations as known in the art. The effective amount is typically determined in appropriately designed clinical trials (dose range studies) and the person versed in the art will know how to properly conduct such trials in order to determine the effective amount. As generally known, the effective amount depends on a variety of factors including the distribution profile within the body, a variety of pharmacological parameters such as half-life in the body, on undesired side effects, if any, on factors such as age and gender, and others.

The term “subject” refers to a mammal, human or non-human.

The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. It should be noted that where various embodiments are described by using a given range, the range is given as such merely for convenience and brevity and should not be construed as an inflexible limitation on the scope of the invention. Accordingly, the description of a range should be considered to have specifically disclosed all the possible sub-ranges as well as individual numerical values within that range.

As used herein, the term “about” is meant to encompass deviation of ±10% from the specifically mentioned value of a parameter, such as temperature, pressure, concentration, etc.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows the effect of drug concentration on mean oil droplet diameter for CBD-loaded SNEDDS formulations diluted with water 1:40, as detailed in Table 3.

FIG. 2 shows pharmacokinetic profiles of CBD (without and with 0.125 mg THC) in different formulations following oral administration.

FIG. 3 shows pharmacokinetic profiles of THC combined with CBD in different formulations following oral administration.

FIG. 4 shows pharmacokinetic profiles of CBD combined with THC (1:1) following oral administration in SNEDDS of tripropionin and MCT oil. The dose was 0.5 mg CBD and 0.5 mg THC per rat.

FIG. 5 shows pharmacokinetic profiles of THC combined with CBD (1:1) following oral administration in SNEDD of tripropionin and MCT oil. The dose was 0.5 mg CBD and 0.5 mg THC/rat.

FIG. 6 shows pharmacokinetic profiles of CBD combined with THC following oral administration in tripropionin SEDD and MCT oil. The dose was 0.125 mg CBD and 2.5 mg THC/rat.

FIG. 7 shows pharmacokinetic profiles of THC combined with CBD oral administration in SNEDD and MCT oil. The dose was 0.125 mg CBD and 2.5 mg THC/rat.

FIGS. 8A-8B are ternary phase diagrams for tributyrin based liquid SEDDS combinations: the nanoemulsion region, droplet size <200 nm (FIG. 8A), and the physically stable region (FIG. 8B).

FIGS. 9A-9C show sedimentation study of formulation TB1 in various aqueous dilutions at room temperature: 1:10 (FIG. 9A), 1:20 (FIG. 9B), and 1:40 (FIG. 9C).

FIGS. 10A-10C show sedimentation study of formulation TB1 in various aqueous dilutions at 4° C.: 1:10 (FIG. 10A), 1:20 (FIG. 10B), and 1:40 (FIG. 10C).

FIGS. 11A-11B show pharmacokinetic profiles of CBD combined with THC following oral administration in tributyrin-based semi-solid and liquid SEDDS at a ratio of CBD to THC of 20:1: CBD plasma concentration (FIG. 11A) and THC plasma concentration (FIG. 11B).

FIGS. 12A-12B show pharmacokinetic profiles of CBD combined with THC following oral administration in tributyrin-based semi-solid SEDDS at different CBD:THC ratios (1:10, 20:1, 1:1 and 1:5): CBD plasma concentration (FIG. 12A) and THC plasma concentration (FIG. 12B).

FIGS. 13A-13B show pharmacokinetic profiles of CBD combined with THC following oral administration of tributyrin at a ratio of CBD to THC of 20:1: CBD plasma concentration (FIG. 13A) and THC plasma concentration (FIG. 13B).

### DETAILED DESCRIPTION OF EMBODIMENTS

Preparation Method of SNEDDS:

The base SNEDDS formulation was prepared by mixing the surfactant and structurants. The required amount of oil was added to this mixture to form the base SNEDDS formulation. 1 ml of the base SNEDDS formulation was taken and required amount of drug (e.g. CBD and/or THC) was added, and the mixture was mixed until a homogenous solution was obtained using magnetic stirring at 1500 rpm for 1 hour to form to a drug-loaded SNEDDS formulation. Upon addition of bi-distilled purified water at different ratios (e.g. from 1:1 up to 1:100), the drug-loaded SNEDDS formulation spontaneously emulsified into fine nanoemulsions.

An exemplary SNEDDS formulation loaded with a mixture of CBD and THC is shown in Table 1. Tripropionin was used as the oil (0), Cremophor RH40 (polyoxyl 40 hydrogenated castor oil) as surfactant (S), PG and PEG 400 as structurants (co-surfactants and/or co-solvents Cs).

TABLE 1
Tripropionin CBD/THC-loaded SNEDDS formulation

| | | | PEG | | | | |
|---|---|---|---|---|---|---|---|
| | TPN | CREM | 400 | PG | Composition | CBD | THC |
| | (mg) | (mg) | (mg) | (mg) | (O:S:Cs) | (mg) | (mg) |
| TPN5 | 200 | 350 | 350 | 350 | (20:35:70) | 50 | 2.5 |

TPN = Tripropionin;
Crem = Cremophor PH-40;
PG = Propylene glycol;
PEG = Polyethylene glycol

Effect of Structurants:

The effect of various structurants, i.e. co-solvents and/or co-surfactants, on the SNEDDS formulation was examined, as shown in Table 2. Formulation F1 and F2 were prepared to compare the efficacy of Glycerin (Glycn) and Propylene Glycol (PG), respectively. F2 containing PG was found to give a faint bluish dilution whereas F1 containing glycerin showed turbidity upon dilution with water in 1:20 ratio and was found to separate into two phases after 24 hours.

The effect of ethanol (EtOH) as co-solvent was observed in formulations F4-F7 at different concentrations. It was observed that the addition of ethanol caused the concentrated SNEDDS to be more turbid on dilution with water in 1:20 ratio, whereas formulation F2 without any ethanol was found to give faint bluish appearance indicating formation of finer nanoemulsion. Hence ethanol was not used as a co-solvent and the formulations were prepared without containing ethanol. This finding is of significance, as ethanol-free oil-based formulations that can self-emulsify upon dilution in water have been, to the best of the inventors knowledge, very difficult to achieve, and may be beneficial for prolonged stability. Such ethanol-free formulations can also be used as formulations for administration to children.

TABLE 2
TPN5 formulation with variable co-surfactant/co-solvent ratios at water dilution of 1:20 and 1:40

| | | | | | | | | | | | Stability | | | |
|---|---|---|---|---|---|---|---|---|---|---|---|---|---|---|
| | TPN | PG | CRM | PEG | Glycn | EtO | CBD | THC | SEDDS | SEDDS | after | Diameter | | ZP |
| | (mg) | (mg) | (mg) | (mg) | (mg) | (μl) | (mg) | (μg) | (1:20) | (1:40) | 24 H | (nm)* | PDI* | (mV) |
| F1 | 200 | 0 | 350 | 350 | 350 | 0 | 0 | 0 | Turbid | Not Chkd | Stable | Not Chkd | | |
| F2 | 200 | 350 | 350 | 350 | 0 | 0 | 0 | 0 | Faint blue | Not Chkd | Stable | 94.46 | 0.788 | −22.4 |
| F3 | 1000 | 1750 | 1750 | 1750 | 0 | 0 | 15 | 750 | Faint blue | Not Chkd | Stable | 695.9 | 0.660 | −24.1 |
| F4 | 200 | 350 | 350 | 350 | 0 | 100 | 0 | 0 | Turbid | Not Chkd | Stable | Not Chkd | | |
| F5 | 200 | 350 | 350 | 350 | 0 | 200 | 0 | 0 | Turbid | Not Chkd | Stable | Not Chkd | | |
| F6 | 200 | 350 | 350 | 350 | 0 | 300 | 0 | 0 | Turbid | Not Chkd | Stable | Not Chkd | | |
| F7 | 200 | 350 | 350 | 350 | 0 | 400 | 0 | 0 | Turbid | Not Chkd | Stable | Not Chkd | | |
| F8 | 200 | 175 | 350 | 525 | 0 | 0 | 0 | 0 | Turbid | Not Chkd | Stable | Not Chkd | | |
| F9 | 200 | 175 | 350 | 525 | 0 | 0 | 0 | 0 | Turbid | Faint blue | Stable | 158.7, | 0.204 | −8.09 |
| | | | | | | | | | | | | 255.3, | 0.308 | |
| | | | | | | | | | | | | 460.6 | 0.433 | |

| | | | | | | | | | | | | |
|---|---|---|---|---|---|---|---|---|---|---|---|---|
| F10 | 200 | 175 | 350 | 525 | 0 | 100 | 0 | 0 | Turbid | Not Chkd | Stable | Not Chkd |
| F11 | 200 | 175 | 350 | 525 | 0 | 200 | 0 | 0 | Turbid | Not Chkd | Stable | Not Chkd |
| F12 | 200 | 175 | 350 | 525 | 0 | 300 | 0 | 0 | Turbid | Not Chkd | Stable | Not Chkd |
| F13 | 200 | 175 | 350 | 525 | 0 | 400 | 0 | 0 | Turbid | Not Chkd | Stable | Not Chkd |
| F14 | 200 | 175 | 350 | 525 | 0 | 500 | 0 | 0 | Turbid | Not Chkd | Stable | Not Chkd |
| F15 | 200 | 175 | 350 | 525 | 0 | 500 | 0 | 0 | Turbid | Faint blue | Stable | Not Chkd |
| F16 | 200 | 0 | 300 | 700 | 0 | 0 | 0 | 0 | Turbid | Not Chkd | Stable | Not Chkd |

*For samples F2 and F3—the size analysis was for a dilution of 1:20; for sample F9, the size analysis was for a dilution of 1:40

Effect of Drug Concentration:

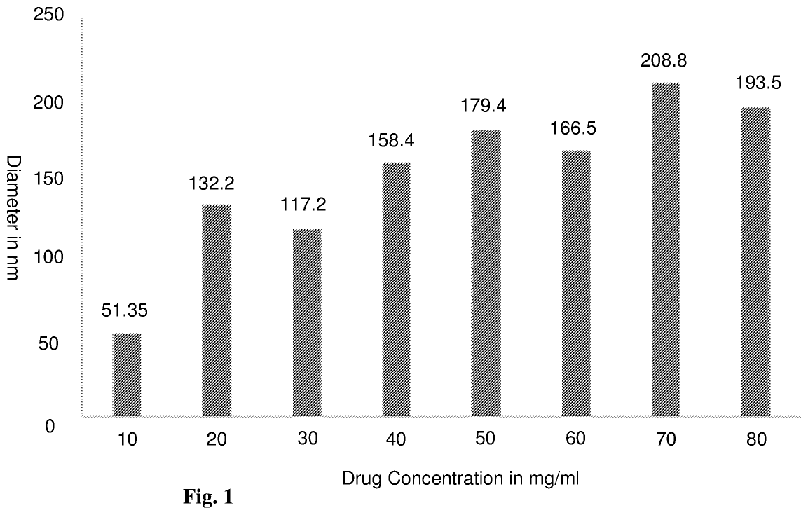

The concentration of drug (CBD) was varied from 10 mg/ml to 80 mg/ml in a SNEDDS formulation comprising 200 mg tripropionin, 350 mg Cremophor RH40, 350 mg PEG 400 and 350 mg PG, as shown in Table 3. It was observed that the particle size and PDI increases with increasing drug concentration. The maximum CBD loading before the diluted formulation formed milky was found to be at 50 mg/ml and 60 mg/ml (TPN5 and TPN6), which showed average diameter of 179.4, 166.5 and PDI of 0.257, 0.331 respectively, as shown also in FIG. 1.

TABLE 3
Effect of drug concentration increase on mean oil droplet diameter and zeta
potential, tripropionin-based SNEDDS diluted with water (1:40 and 1:5)

| | CBD | | | | | | | | | | |
|---|---|---|---|---|---|---|---|---|---|---|---|
| | (mg | SEDDS (1:40) | | | | | SEDDS (1:5) | | | | |
| | per | | Size | | | ZP | | Size | | | ZP |
| | ml) | Appearance | (nm) | Distribution | PDI | (mV) | Appearance | (nm) | Distribution | PDI | (mV) |
| TPN1 | 10 | Faint blue | 51.35 | P1 = 269.2 (61.3%) | 0.789 | −19.4 | −19.4 | 90.38 | P1 = 237.9 (79.6%) | 0.622 | −15.4 |
| | | | | P2 = 21.90 (35.5%) | | | | | P2 = 18.29 (18.4%) | | |
| | | | | P3 = 4152 (3.3%) | | | | | P3 = 4655 (2.0%) | | |
| TPN2 | 20 | Faint blue | 132.3 | P1 = 132.3 (92.9%) | 0.548 | −22.9 | −22.9 | 109.1 | P1 = 167.5 (86.4%) | 0.445 | −15.0 |
| | | | | P2 = 18.84 (7.1%) | | | | | P2 = 18.23 (8.2%) | | |
| | | | | | | | | | P3 = 4045 (5.4%) | | |
| TPN3 | 30 | Faint blue | 117.2 | P1 = 160.5 (97.5%) | 0.276 | −20.7 | −20.7 | 174.9 | P1 = 226.3 (95%) | 0.294 | −13.4 |
| | | | | P2 = 15.59 (2.5%) | | | | | P2 = 3922 (5%) | | |
| TPN4 | 40 | Faint blue | 158.4 | P1 = 189.8 (97.3%) | 0.242 | −21.6 | −21.6 | 175.4 | P1 = 213.4 (98.0%) | 0.259 | −14.1 |
| | | | | P2 = 4675 (2.7%) | | | | | P2 = 5017 (2.0%) | | |
| TPN5 | 50 | Slightly | 179.4 | P1 = 236.2 (98.7%) | 0.257 | −30.3 | −30.3 | 192.0 | P1 = 224.7 (96.3%) | 0.266 | −16.5 |
| | | milky | | P2 = 4980 (1.3%) | | | | | P2 = 4867 (3.7%) | | |
| TPN6 | 60 | Milky | 166.5 | P1 = 249.6 (98.9%) | 0.331 | −26.0 | −26.0 | 178.1 | P1 = 214.7 (99%) | 0.239 | −16.9 |
| | | | | P2 = 4584 (1.1%) | | | | | P2 = 5235 (1%) | | |
| TPN7 | 70 | Milky | 208.8 | P1 = 255.8 (92.3%) | 0.381 | −33.6 | −33.6 | 230.7 | P1 = 291.8 (95.2%) | 0.465 | −24.0 |
| | | | | P2 = 4475 (7.7%) | | | | | P2 = 5108 (4.8%) | | |
| TPN8 | 80 | Milky | 193.5 | P1 = 223.7 (96.3%) | 0.324 | −33.8 | −33.8 | 297.0 | P1 = 206.9 (70.9%) | 0.405 | −23.1 |
| | | | | P2 = 5527(2.3%) | | | | | P2 = 612.3 (27.1%) | | |
| | | | | P3 = 34.42 (1.5%) | | | | | P3 = 5511 (2.0%) | | |

Effect of the Dilution Ratio

The effect of water dilution on the size and polydispersity of the TPN5 formulation droplets in the resulting nanoemulsion was observed at various dilutions, as shown in Table 4. The size and polydispersity do not seem to be significantly dependent on the dilution ratio, as the values remained more or less similar at different ratios.

TABLE 4
Effect of water dilution ratio for formulation TPN5 on
mean oil droplet diameter

| Dilution ratio (v/v) | Appearance | Size (nm) | PDI |
|---|---|---|---|
| 1:1 | Milky | 276.8 | 0.418 |
| 1:2 | Milky | 230.4 | 0.348 |
| 1:3 | Milky | 202.4 | 0.266 |
| 1:4 | Milky | 194.8 | 0.312 |
| 1:5 | Milky | 201.0 | 0.375 |
| 1:10 | Milky | 238.7 | 0.334 |
| 1:20 | Milky | 209.1 | 0.313 |
| 1:40 | Bluish | 199.2 | 0.325 |
| 1:80 | Bluish | 195.9 | 0.328 |
| 1:100 | Bluish | 216.6 | 0.432 |

Physical Stability of the Formulations at Room Temperature.

The physical stability of formulations TPN5 (used in the pharmacokinetic study to be described below) was evaluated at room temperature. The stability has been evaluated in terms of appearance, particle size distribution and zeta potential. The observations have been made for Day 1, 2, 3 and 7, as shown in Tables 5-1 and 5-2 (Table 5-1 shows the results for an oil concentrate diluted with water on day 0 to form SEDDS and samples taken at every measurement point—designated as “originally reconstituted”; while Table 5-2 shows the results for samples of oil concentrate diluted with water at each of the measurement points—designated as “freshly reconstituted”). For formulation TPN5, it was observed that there was no significant change in properties in both the dilution ratios of 1:40 and 1:5. However, for F3 with CBD concentration of 2.5 mg/ml combined with 0.125 mg/ml THC, the size and PDI were variable and not stable.

TABLE 5-1
Physical Stability at Room Temperature of TPN5 [CBD = 50 mg/ml]
over 7 days following water dilution of the oil concentrate (1:40 and 1:5)
[“originally reconstituted”]

| | | Size (nm) | Size (nm) | Zeta potential |
|---|---|---|---|---|
| Day | Appearance | (1:40) | (1:5) | (mV) |
| 0 | Bluish milky | 192.4 | 192.4 | −27.5 |
| | | PDI = 0.362 | PDI = 0.362 | |
| 1 | Bluish milky | 199.8 | 201.8 | −24.1 |
| | | PDI = 0.353 | PDI = 0.336 | |
| 2 | Bluish milky | 197.8 | 200.0 | −33.1 |
| | | PDI = 0.340 | PDI = 0.316 | |
| 3 | Bluish milky | 190.3 | 192.2 | −26.4 |
| | | PDI = 0.345 | PDI = 0.291 | |
| 7 | Bluish milky | 192.1 | 209.9 | −25.3 |
| | | PDI = 0.273 | PDI = 0.326 | |

TABLE 5-2
Physical Stability at Room Temperature of TPN5 [CBD = 50 mg/ml]
over 7 days following water dilution of the oil concentrate (1:40 and 1:5)
[“freshly reconstituted”]

| | | Size (nm) | Size (nm) | Zeta potential |
|---|---|---|---|---|
| Day | Appearance | (1:40) | (1:5) | (mV) |
| 1 | Bluish milky | 193.1 | 208.8 | −29.2 |
| | | PDI = 0.274 | PDI = 0.342 | |
| 2 | Bluish milky | 189.5 | 209.9 | −36.3 |
| | | PDI = 0.331 | PDI = 0.354 | |
| 3 | Bluish milky | 195.7 | 197.5 | −30.3 |
| | | PDI = 0.354 | PDI = 0.270 | |
| 7 | Bluish milky | 182.5 | 214.1 | −28.6 |
| | | PDI = 0.311 | PDI = 0.342 | |

Long-Term Physical Stability of the Formulations

The long-term physical stability of formulation TPN5 at accelerated conditions was assessed at room temperature (25° C.), 4° C. and 37° C. The stability was evaluated in terms of appearance, particle size distribution, Zeta potential and drug content, as shown in Tables 6-1 to 6-3. No significant changes were observed in all evaluation parameters.

TABLE 6-1
Accelerated stability for TPN5 at room temperature

| | | | Zeta potential | CBD content | THC content |
|---|---|---|---|---|---|
| Day | Size (nm) | PDI | (mV) | (mg) | (mg) |
| 0 | 176.9 | 0.324 | −31.3 | 48.82 | 1.421 |
| | 168.9 | 0.289 | −27.6 | 46.14 | 1.409 |
| | 182.2 | 0.340 | −23.1 | 46.36 | 1.500 |
| 15 | 188.6 | 0.343 | −29.3 | 45.96 | 1.724 |
| | 168.52 | 0.252 | −28.9 | 55.38 | 2.100 |
| | 166.2 | 0.308 | −34.3 | 43.64 | 1.612 |
| 30 | 205.3 | 0.360 | −21.7 | 45.01 | 1.559 |
| | 173.0 | 0.325 | −22.7 | 45.18 | 1.553 |
| | 174.3 | 0.331 | −21.8 | 44.61 | 1.564 |
| 45 | 201.8 | 0.347 | −22.5 | 46.66 | 1.534 |
| | 182.3 | 0.335 | −20.1 | 53.28 | 1.714 |
| | 175.3 | 0.355 | −20.5 | 51.54 | 1.549 |
| 60 | 190.0 | 0.314 | −17.9 | 48.85 | 1.59 |
| | 172.8 | 0.372 | −20.3 | 47.01 | 1.66 |
| | 168.5 | 0.341 | −19.6 | 43.393 | 1.58 |

TABLE 6-2
Accelerated stability for TPN5 at 4° C.

| | | | Zeta potential | CBD content | THC content |
|---|---|---|---|---|---|
| Day | Size (nm) | PDI | (mV) | (mg) | (mg) |
| 15 | 189.0 | 0.319 | −31.7 | 45.18 | 1.884 |
| | 187.8 | 0.291 | −25.4 | 50.38 | 1.896 |
| | 168.4 | 0.332 | −27.0 | 43.06 | 1.804 |
| 30 | 171.4 | 0.327 | −19.5 | 47.36 | 1.628 |
| | 183.7 | 0.290 | −25.4 | 44.74 | 1.324 |
| | 194.0 | 0.398 | −20.1 | | 1.043 |
| 45 | 196.0 | 0.328 | −21.9 | 41.08 | 1.610 |
| | 180.7 | 0.339 | −20.7 | 45.36 | 1.430 |
| | 199.4 | 0.354 | −22.0 | 47.20 | 1.467 |
| 60 | 160.7 | 0.261 | −18.4 | 43.07 | 1.56 |
| | 169.8 | 0.299 | −18.5 | 45.23 | 1.64 |
| | 178.7 | 0.313 | −17.7 | 39.09 | 1.45 |

TABLE 6-3
Accelerated stability for TPN5 at 37° C.

| | | | Zeta potential | CBD content | THC content |
|---|---|---|---|---|---|
| Day | Size (nm) | PDI | (mV) | (mg) | (mg) |
| 15 | 179.9 | 0.275 | −31.6 | 46.20 | 1.692 |
| | 176.5 | 0.278 | −29.4 | 40.34 | 1.464 |
| | 186.2 | 0.351 | −25.2 | | 1.621 |
| 30 | 182.1 | 0.333 | −17.9 | 47.24 | 1.656 |
| | 179.5 | 0.382 | −19.7 | 46.52 | 1.605 |
| | 178.1 | 0.360 | −22.9 | | 1.322 |
| 45 | 182.2 | 0.328 | −22.6 | 57.22 | 1.468 |
| | 180.5 | 0.312 | −18.1 | 65.40 | 1.468 |
| | 181.1 | 0.265 | −19.8 | 58.12 | 1.490 |
| s60 | 177.1 | 0.276 | −16.2 | 45.93 | 1.57 |
| | 167.5 | 0.349 | −19.5 | 46.28 | 1.62 |
| | 164.4 | 0.260 | −19.0 | 46.16 | 1.59 |

Comparative Reference 1: MCT-Based Formulation

MCT (medium chain triglyceride) is a commonly used oil component in many emulsion-based commercial formulations. MCT was assessed for suitability as a single oil component, together with Cremophor RH40 as surfactant, and PG, Glycerin, PEG 400 and ethanol as co-surfactants and co-solvents. The mixtures were diluted. In order to obtain a water dilution of 1:20, 100μl of the mixture were added to 2 ml of double distilled water. SEDDS with faint bluish appearance were observed for MCT-to-surfactants ratios of 1:9 to 1:5. In preparations with higher ratios i.e. 1:4 to 1:1, the SEDDS formed after dilution were turbid, as shown in Table 7.

Further, following 24 hours standing at room temperature conditions, phase separation was observed in all the MCT-based formulations. Hence the preparation of this series of SEDDS was not further considered for evaluation.

TABLE 7
MCT-based formulations at water dilution of 1:20

| | | | | | | | | | Sta- |
|---|---|---|---|---|---|---|---|---|---|
| | | | | | | | | | bility |
| | MCT: | PG | CRM | PEG | Glycn | EtOH | MCT | SEDDS | after |
| | S/CS | (mg) | (mg) | (mg) | (mg) | (μl) | (μl) | (1:20) | 24 H |
| S1 | 1:9 | 500 | 500 | 0 | 500 | 200 | 188.8 | Good | Un- |
| | | | | | | | | | stable |
| S2 | 1:8 | 500 | 500 | 0 | 500 | 200 | 212.5 | Good | Un- |
| | | | | | | | | | stable |
| S3 | 1:7 | 500 | 500 | 0 | 500 | 200 | 242 | Good | Un- |
| | | | | | | | | | stable |
| S4 | 1:6 | 500 | 500 | 0 | 500 | 200 | 283 | Good | Un- |
| | | | | | | | | | stable |
| S5 | 1:5 | 500 | 500 | 0 | 500 | 200 | 340 | Good | Un- |
| | | | | | | | | | stable |
| S6 | 1:4 | 500 | 500 | 0 | 500 | 200 | 425 | Turbid | Un- |
| | | | | | | | | | stable |
| S7 | 1:3 | 500 | 500 | 0 | 500 | 200 | 566.6 | Turbid | Un- |
| | | | | | | | | | stable |
| S8 | 1:2 | 500 | 500 | 0 | 500 | 200 | 850 | Turbid | Un- |
| | | | | | | | | | stable |
| S9 | 1:1 | 500 | 500 | 0 | 500 | 200 | 700 | Turbid | Un- |
| | | | | | | | | | stable |
| S10 | 1:21 | 0 | 350 | 350 | 350 | 0 | 50 | Turbid | Un- |
| | | | | | | | | | stable |

Comparative Reference 2: Peceol-Based Formulation

Peceol was another oil tested for suitability to form stable CBD formulations. Peceol was used instead of tripropionin as an oil component in a formulation containing a surfactant mixture of Cremophor EL and Tween 20, and PG and ethanol. Formation of SEDDS was observed for dilution of 1:40 (50μl of formulation in 2 ml of double distilled water), and effect of increasing drug concentration of the mean droplet size and zeta potential was assessed. The mean droplet size decreased with increasing drug concentration, showing average diameter of 162.7 nm at 10 mg/ml drug concentration to 122.8 nm at 50 mg/ml. However, the preparations were less homogenous, showing multiple peaks and high polydispersity of 0.414 to 0.536 compared to the formulations of tripropionin, as shown in Table 8.

TABLE 8
Peceol-based formulations at water dilution of 1:20

| | Peceol | CRM | PEG | Tween20 | EtOH | Composition | CBD | SEDDS | Size | | | ZP |
|---|---|---|---|---|---|---|---|---|---|---|---|---|
| | (mg) | (mg) | (mg) | (mg) | (mg) | (O:s:Cs:Csv) | (mg) | (1:40) | (nm) | Distribution | PDI | (mV) |
| PAC1 | 300 | 250 | 100 | 250 | 100 | 30:50:10:10 | 10 | Slightly | 162.7 | P1 = 231.5 (87.5%) | 0.476 | −31.4 |
| | | | | | | | | milky | | P2 = 28.46 (7.4%) | | |
| | | | | | | | | | | P3 = 4723 (5.1%) | | |
| PAC2 | 300 | 250 | 100 | 250 | 100 | 30:50:10:10 | 20 | Slightly | 170.7 | P1 = 255.1 (74.4%) | 0.518 | −28.7 |
| | | | | | | | | milky | | P2 = 3306 (13.6%) | | |
| | | | | | | | | | | P3 = 33.23 (12.1%) | | |
| PAC3 | 300 | 250 | 100 | 250 | 100 | 30:50:10:10 | 30 | Slightly | 144.1 | P1 = 320.0 (88.1%) | 0.536 | −27.1 |
| | | | | | | | | milky | | P2 = 30.39 (9.2%) | | |
| | | | | | | | | | | P3 = 4807 (2.3%) | | |
| PAC4 | 300 | 250 | 100 | 250 | 100 | 30:50:10:10 | 40 | Slightly | 135.4 | P1 = 478.5 (100%) | 0.510 | −29.0 |
| | | | | | | | | milky | | | | |
| PAC5 | 300 | 250 | 100 | 250 | 100 | 30:50:10:10 | 50 | Slightly | 122.8 | P1 = 181.3 (96.8%) | 0.414 | |
| | | | | | | | | milky | | P2 = 4729 (3.0%) | | |

Pharmacokinetic Studies

The pharmacokinetic evaluation was carried out using the tripropionin formulations with the marked advantage of practically no ethanol, making such formulations suitable for use in children and also making them stable over 7 days without change in the physicochemical properties of the formulations.

Tripropionin-based formulations for Pharmacokinetic study were prepared using system of Cremophor RH40 as surfactant, and PG and PEG 400 as the structurants, in the ratio of 200 parts oil, and 350 parts each of PG, PEG and Cremophor RH 40. 10 ml of the base formulation was taken and required amount of drug (CBD 25 mg and THC 1.25 mg) was added to obtain a final drug concentration of 2.5 mg/ml CBD and 0.125 mg/ml THC. The drug-loaded SNEDDS formulation was then homogenized using magnetic stirring at 1500 rpm for 1 hour.

Pharmacokinetic Studies of CBD and THC SNEDDS on SD Rats

Study Protocol

Five animals were used in each treatment group, blood samples were withdrawn from the tail at time points: 0, 0.5, 1, 2, 4, 6, 10, 24 h.

Each 3 animals received a tripropionin- or MCT-based formulation containing CBD and CBD/THC at various ratios, dosed by gavage every day of the experiment. 1 ml of formulation followed by gavage of 1 ml of water to allow dispersion in the GI tract of the various oil formulations. The SNEDDS formulations of CBD were compared to a CBD olive oil marketed product.

Plasma was separated by centrifugation (4000 rpm, 4° C., 10 minutes) and stored at −80° C. till the day of analysis using LC-MS/MS.

Determination of CBD and THC by LC-MS/MS

The analytical studies were carried out by the Mass Spectrometry Unit of the Institute for Drug Research of the School of Pharmacy, Faculty of Medicine, The Hebrew University of Jerusalem.

Materials: LC/MS-grade Acetonitrile (ACN), Methanol (MeOH) and water were purchased from Biolab Ltd. (Jerusalem, Israel). Formic acid (FA) was purchased from J. T. Baker (USA).

UHPLC instrument: The chromatography was performed under reverse phase conditions using a Thermo Scientific, San Jose, Calif., USA which includes and Dionex Pump with degasser module and an Accela Autosampler. The chromatographic separations were performed on a Kinetex™ (Phenomenex, Torrance, Calif., USA) column (EVO C18, 2.6 μm particle size, 100 Å pore size, 50×2.1 mm), protected by a SecurityGuard™ (Phenomenex, Torrance, Calif., USA) cartridges (C18, 4×2.1 mm). The injection volume was 25 μL, the oven temperature was maintained at 40° C. and the autosampler tray temperature was maintained at 4° C.

UHPLC conditions: The chromatographic separation was achieved using a linear gradient program at a constant flow rate of 0.3 mL/min over a total run time of 9 min. An outline of the mobile phase gradient program is summarized in Table 9. The column effluent was diverted away from the MS during the first 0.9 min and last 2.0 min of the run. A mixture of water:MeOH (1:1) was used for washing the needle prior to each injection cycle. All samples were analyzed in duplicate.

TABLE 9
Gradient program: Solvent A is 0.1% FA in water and solvent B is ACN.

| Time, min | Solvent A, % | Solvent B, % |
|---|---|---|
| 0.0 | 40 | 60 |
| 4.0 | 5 | 95 |
| 6.0 | 5 | 95 |
| 6.39.0 | 40 | 60 |
| | 40 | 60 |

MS/MS conditions: CBD, THC and CBG (IS) were detected by a TSQ Quantum Access Max mass spectrometer in positive ion mode using electron spray ionization (ESI) and multiple reaction monitoring (MRM) mode of acquisition. The high-purity nitrogen gas (15 L min-1), used as sheath and auxiliary gases, was generated using a Parker nitrogen generator (Parker Hannifin ltd., Gateshead, Tyne and Wear, England). 99.999% pure argon (Moshalion, Jerusalem, Israel) was used as collision gas (1.5 mTorr). Optimal detection conditions were determined by constant infusion of 200 ng/mL solutions of the analytes in 9:1 ACN:water using the integrated syringe pump (10 μL/min). Transitions were selected and their settings were determined using TSQ Tune Software (Thermo Scientific, San Jose, Calif., USA). The spray voltage, sheath and auxiliary gas were set at 5000V, 30 and 60 (arbitrary units), respectively. The capillary transfer tube temperature was set at 220° C.; the tube lens was set at 91V for CBD and THC and 71V for CBG. The vaporizer temperature within the H-ESI source was 450° C. The scan time was 50 ms, scan width 0.1 m/z, Q1 and Q3 peak width of 0.7 Da (unit). TSQ Tune Software (Thermo Scientific, San Jose, Calif., USA) was used for the optimization of tuning parameters.

The molecular ions of the compounds [M+H]+ were selected in the first mass analyzer and fragmented in the collision cell followed by detection of the products of fragmentation in the second analyzer. The following transitions were monitored:

CBD: m/z 315→193 (quantifier), collision energy (CE) 20V and m/z 315→123 (qualifier), CE 32V, retention time (RT) 2.6 min.

THC: m/z 315→193 (quantifier), CE 20V and m/z 315→123 (qualifier), CE 32V, RT 3.5 min.

CBG: m/z 317→193 (quantifier), collision energy (CE) 18V and m/z 317→123 (qualifier), CE 32V, retention time (RT) 2.6 min.

Data acquisition and processing were carried out using the Xcalibur program (Thermo Scientific, San Jose, Calif., USA). Quantitative calibration (1-500 ng/ml) was performed before every batch of samples. The calibration curves were created using peak-area ratios (analyte versus internal standard). The calibration curve (y=a+bx) was obtained by weighted (1/y) linear least-squares regression of the measured peak-area ratios (y) CBD/CBG (or THC/CBG) versus the concentration of CBD (or THC) added to the plasma (x). The limit of quantification (LOQ) was 0.5 ng/mL for CBD and THC.

Results

From the systematic review on the pharmacokinetic (pk) of CBD in humans [22] only 24 out of 792 retrieved included pharmacokinetic parameters in humans reflecting the paucity of the pharmacokinetic data and information on the CBD irrespective of the route of administration. Bioavailability following smoking was 31% of the dose, the oral bioavailability including animal studies was 13-19% [23]. Some studies in humans showed values as low as 6% for oral administration of oil solutions [24]. The reason for such low oral bioavailability is the lipophilicity of the cannabinoids and particularly THC and CBD, which undergo extensive first pass metabolism and their metabolites are mostly excreted via the kidneys [25]. It was also reported that plasma and brain concentrations are dose-dependent in animals, and bioavailability is increased with various lipid formulations [26]. Moreover, despite the breadth of use of CBD in humans, there is little data to date on its pharmacokinetics. Oromucosal spray, either buccal, sublingual, or oropharyngeal administration, resulted in mean Cₘₐₓ between 2.5 and 3.3 ng/mL and mean Tₘₐₓ between 1.64 and 4.2 h. Sublingual drops resulted in similar Cₘₐₓ of 2.05 and 2.58 ng/mL and Tₘₐₓ of 2.17 and 1.67 h, respectively using CBD doses of 10 or 20 mg.

In the present study, CBD alone or in various combinations with THC were prepared in SNEDDS and evaluated for the contribution of specific formulation on the potential to enhance the oral absorption of CBD alone or in combination with THC. In addition, the absorption of THC was also determined and was evaluated as a function of the CBD concentration and the type of formulation.

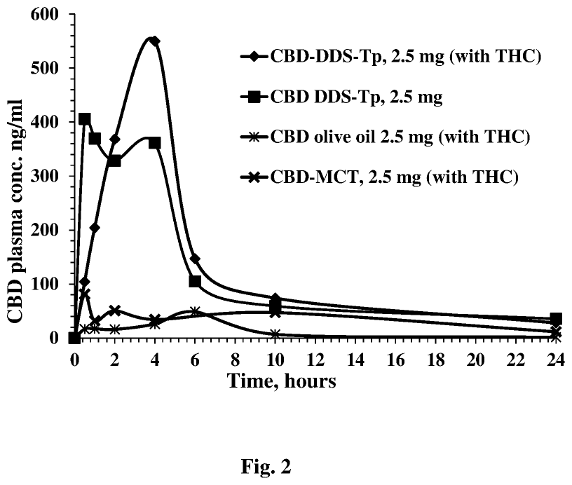

The mean pharmacokinetic profiles of the CBD at a fixed dose of 2.5 mg per rat, with and without THC, at various doses are presented in FIG. 2 and the values of the various pK parameters are reported in Table 10-1. It was noted that the highest plasma levels were elicited by the two tripropionin (Tp)-SNEDDS, with and without THC.

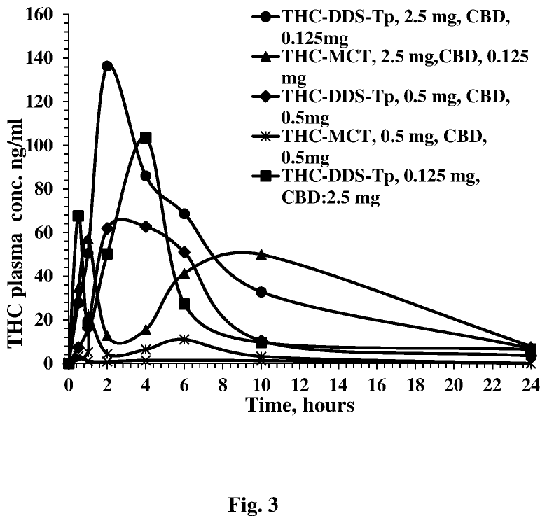

A more moderate trend was observed for THC at a dose of 2.5 mg as observed in FIG. 3 and Table 10-2 summarizes all the mean pharmacokinetic parameters' values of the various THC doses and formulations.

TABLE 10-1
Pharmacokinetics parameters of CBD following oral administration in
different formulation and doses of CBD alone or combined with THC

| | | | | | | Volume of |
|---|---|---|---|---|---|---|
| | Cmax | Tmax | T1/2 | AUC | Clearance | distribution |
| Formulation | (ng/ml) | (hours) | (hours) | (ng/ml × h) | (ml/h) | (ml) |
| CBD-DDS-Tp, 2.5 mg | 550 | 4 | 8 | 3317 | 754 | 8744 |
| (0.125 mg THC) | | | | | | |
| CBD-DDS-Tp, 2.5 mg | 406 | 1 | 13 | 3349 | 746 | 13881 |
| CBD-olive oil, 2.5 mg | 49 | 6 | 4 | 280 | 8924 | 45518 |
| (0.125 mg THC) | | | | | | |
| CBD-MCT, 2.5 mg | 178 | 6 | 5 | 1210 | 2066 | 14932 |
| (0.125 mg THC) | | | | | | |
| CBD-DDS, 0.5 mg | 85 | 1 | 9 | 685 | 730 | 9220 |
| (0.5 mg THC) | | | | | | |
| CBD-MCT, 0.5 mg | 49 | 1 | 4 | 183 | 2728 | 14449 |
| (0.5 mg THC) | | | | | | |
| CBD-DDS, 0.125 mg | 12 | 1 | 79 | 1131 | 111 | 12646 |
| (2.5 mg THC) | | | | | | |
| CBD-MCT, 0.125 mg | 14 | 1 | 179 | 2141 | 58 | 15151 |
| (2.5 mg THC) | | | | | | |

TABLE 10-2
Effect of decreasing doses of THC and increasing dose of CBD per rat on
the THC plasma pharmacokinetics parameters following oral administration in
different formulations and doses

| | | | | | | Volume of |
|---|---|---|---|---|---|---|
| | Cmax | Tmax | T1/2 | AUC | Clearance | distribution |
| Formulation | (ng/ml) | (hours) | (hours) | (ng/ml × h) | (ml/h) | (ml) |
| THC-DDS-Tp, 2.5 mg | 136 | 2 | 5.5 | 920 | 2567 | 22077 |
| (0.125 mg CBD) | | | | | | |
| THC-DDS-Tp, 0.5 mg | 63 | 4 | 4.8 | 476 | 1008 | 7743 |
| (0.5 mg CBD) | | | | | | |
| THC-DDS-Tp, 0.125 mg | 103 | 4 | 6.3 | 516 | 223 | 1975 |
| (2.5 mg CBD) | | | | | | |
| THC-MCT, 2.5 mg | 57 | 1 | 6.7 | 647 | 3432 | 44600 |
| (0.125 CBD) | | | | | | |
| THC-MCT, 0.5 mg | 23 | 1 | 5.4 | 67 | 5200 | 46007 |
| (0.5 mg CBD) | | | | | | |
| THC-MCT, 0.125 mg | 3.4 | 0.5 | 3 | 18 | 6952 | 30478 |
| (2.5 mg CBD) | | | | | | |

The AUC values elicited by the oral administration of THC and CBD at different formulations and combinations were subjected to the statistical analysis using unpaired t-test of all the plasma of THC and CBD levels between the animals in the same group and between the various groups in the animal experiments. The results are presented in Tables 11-1 and 11-2, respectively. It should be noted that the mean AUC values in the statistical test are different from the values in the Tables 10-1 and 10-2, the AUC values of which were calculated from the mean plasma levels of all the animals in the same group followed by the calculation of the mean AUC for the entire group whereas in the statistical test, the AUC of each animal in the same group was first calculated and the mean value is presented in Table 11-1 and 11-2 for THC and CBD respectively with the standard deviation within the group.

It could be clearly observed in Table 11-1 that all the SNEDDS-Tp groups are significantly different from the respective MCT groups despite the high standard deviation within the groups. The difference in the magnitude between the respective groups in Table 11-1 refer to the same dose orally administered to the animals and not to the normalized AUC value per mg THC orally administered.

TABLE 11-1
Statistical analysis of THC groups (the analysis was carried out
following individual calculation of each AUC for THC per animal
in the same group following oral administration of Tp-SNEDDS and
comparison of the mean AUC values for THC with the respective
MCT group)

| Group | Formulation | AUC (ng/ml × h) |
|---|---|---|
| 1 | THC-DDS-Tp, 0.125 mg | 451.915 ± 345.75 |
| | (2.5 mg CBD) | (11.1 folds compared to 4) |
| 2 | THC-DDS-Tp, 0.5 mg | 476.03 ± 476.3 |
| | (0.5 mg CBD) | (5.73 folds compared to 5) |
| 3 | THC-DDS-Tp, 2.5 mg | 1080 ± 543 |
| | (0.125 mg CBD) | (2.17 folds compared to 4) |
| 4 | THC-MCT, 0.125 mg (2.5 CBD) | 40.63 ± 44.25 |
| 5 | THC-MCT, 0.5 mg (0.5 mg CBD) | 83.08 ± 62.68 |
| 6 | THC-MCT, 2.5 mg (0.125 mg CBD) | 497.04 ± 374 |

TABLE 11-2
Statistical analysis of CBD groups (the analysis was carried out
following individual calculation of each AUC for CBD per animal
in the same group following oral administration of Tp-SNEDDS and
comparison of the mean AUC values for CBD with the respective
MCT group)

| Group | Formulation | AUC (ng/ml × h) |
|---|---|---|
| 1 | CBD-DDS-Tp, 2.5 mg (0.125 mg THC) | 3047.87 ± 1564.07 |
| 2 | CBD-DDS-Tp, 2.5 mg | 2882.9 ± 1659 |
| 3 | CBD-DDS-Tp, 0.5 mg (0.5 mg THC) | 618.7 ± 320.23 |
| 4 | CBD-MCT, 2.5 mg (0.125 mg THC) | 900.34 ± 458 |
| 5 | CBD-MCT, 0.5 mg (0.5 mg THC) | 187.7 ± 151.87 |
| 6 | CBD-olive oil, 2.5 mg (0.125 mg THC) | 280.83 ± 197.9 |

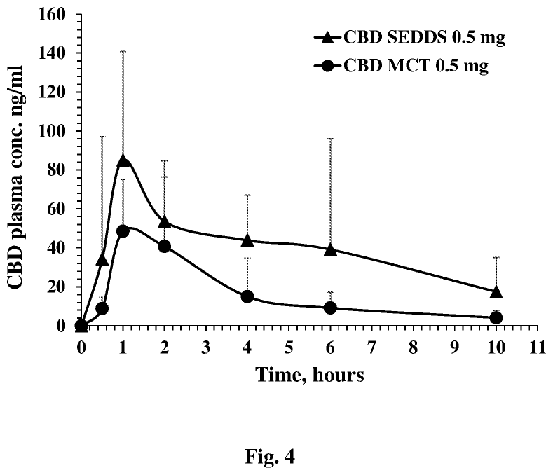

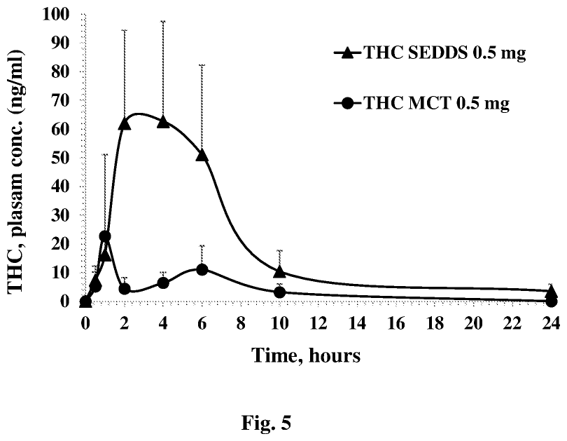

It can also be noted from the results presented in Table 11-1 that THC at a dose of 2.5 mg combined with a small dose of 0.125 mg of CBD in Tp-SNEDDS elicited a high AUC value of 1080±543 ng/ml×h, whereas in MCT oil the same combination elicited 46% of the AUC value (497/1080×100=46%). However, when the dose of THC is decreased and the dose of CBD is increased in parallel, then, the relative absorption of THC is improved as noted, since if the AUC value per mg of THC administered is normalized, then, it can be observed that when the THC:CBD dose combined was 2.5:0.125 mg (ratio 20:1) —the AUC value per mg of THC was 432 ng/ml×h; at a combined dose of 0.5:0.5 mg (ratio 1:1), the AUC value per mg was 952 ng/ml×h; and when the ratio is 1:20 for THC:CBD, 0.125 mg:2.5 mg, then the AUC value per mg increased markedly to 3616 ng/ml×h. The respective values for the MCT oil are 198, 166 and 325 ng/ml×h. This dramatic change in the effect of CBD increasing dose on the normalized bioavailability of THC in the SNEDDS compared to the MCT formulations is, without wishing to be bound by theory, attributed to the SNEDD which enhanced significantly the absorption of CBD, protecting the THC from being degraded in the enterocytes in the liver since it acts as a substrate for the isoenzymes inhibiting the metabolism of THC and allowing higher plasma levels and Tₘₐₓ values. See also FIGS. 3-5.

The effect of decreasing dose of CBD and increasing dose of THC per rat on the CBD plasma pharmacokinetics parameters following oral administration in different formulations is depicted in Table 12.

TABLE 12
Effect of decreasing doses of THC and increasing dose of CBD per rat on the
CBD plasma pharmacokinetics parameters following oral administration in
different formulations and doses

| | | | | | | Volume of |
|---|---|---|---|---|---|---|
| | Cmax | Tmax | T1/2 | AUC | Clearance | distribution |
| Formulation | (ng/ml) | (hours) | (hours) | (ng/ml × h) | (ml/h) | (ml) |
| CBD-DDS-Tp, 2.5 mg | 550 | 4 | 8 | 3317 | 754 | 8744 |
| (0.125 mg THC) | | | | | | |
| CBD-DDS-Tp, 0.5 mg | 85 | 1 | 9 | 685 | 730 | 9220 |
| (0.5 mg THC) | | | | | | |
| CBD-DDS-Tp, 0.125 mg | 12 | 1 | 79 | 1131 | 111 | 12646 |
| (2.5 mg THC) | | | | | | |
| CBD-MCT, 0.5 mg | 49 | 1 | 4 | 183 | 2728 | 14449 |
| (0.5 THC) | | | | | | |
| CBD-MCT, 0.125 mg | 14 | 1 | 179 | 985 | 58 | 15151 |
| (2.5 mg THC) | | | | | | |

Effect of THC Combination

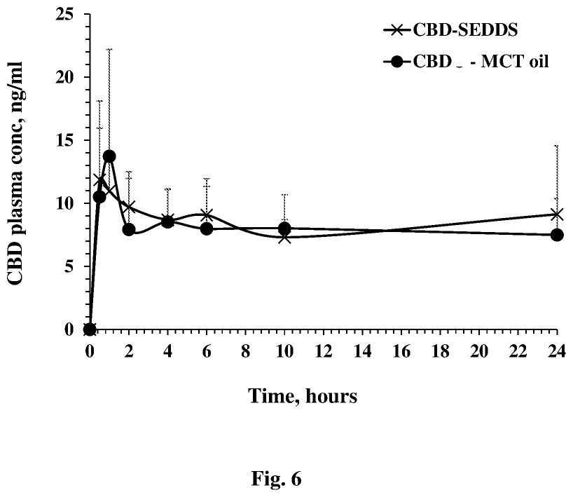

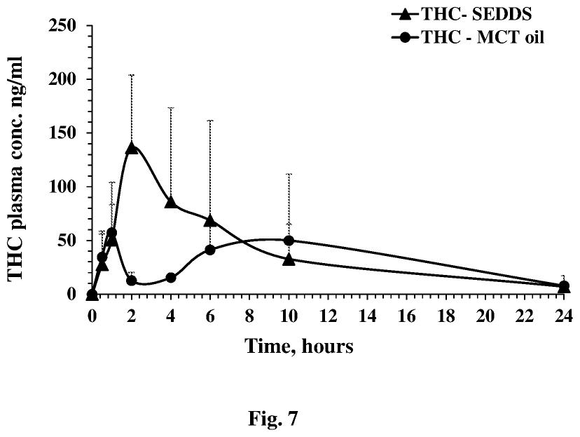

It can be noted from FIG. 6 that the value of CBD plasma elicited at a dose of 0.125 mg of CBD and 2.5 mg of THC did not enhance the absorption of CBD compared to the MCT formulation, meaning that at least a dose of 0.5 mg of CBD is needed to elicit enough plasma levels—in contrary to THC which is present in the formulation at the high dose of 2.5 mg (FIG. 7), and further elicited much more higher absorption profile than with the MCT formulation.

With the combination of 1:1, i.e. 0.5:0.5 mg absorbed, the AUC value for CBD was 618.7±320.23 ng/ml×h for the SNEDDS formulation, compared to 187.7±151.87 ng/ml×h for the MCT formation, showing an improvement in the bioavailability of the same combination and dose of 4-folds. For the 2.5 mg in combination with 0.125 mg dose, at 20:1 ratio, the improvement in bioavailability was 3.4-folds in favor of the SNEDDS (Table 11-2). In contrast with the THC absorption effect, here the AUC value normalized per mg CBD is not affected by the increase of THC in the formulation for the 0.125 mg THC with a ratio of 20:1, the value is 1219 ng/ml×h and for the ratio of 1:1 (0.5:05 mg) the value is 1236 ng/ml×h, confirming the hypothesis that CBD rather protects THC during the absorption path than THC protecting CBD.

Further, the Tp-SNEDDS of CBD was compared to the Olive oil marketed formulation; here, again, there is marked significance in the absorption of CBD compared with the olive oil solution and 2.66-fold in favor of the Tp-SNEDDS (Tables 11-1 and 11-2).

Semi-Solid SEDDS

Preparation for Semi-Solid SEDDS

Typically, SEDDS formulations are liquid, making them challenging to pack into soft gel capsules. Hence, formulations based on tributyrin as an oil component were developed, with the aim of increasing viscosity of the SEDDS to enable stable packaging in soft gel or hard capsules.

Tributyrin liquid SEDDS were first prepared and evaluated. The increase in viscosity of these formulations was achieved by adding either PEG 4000 or PEG 8000. The semi-solid SEDDS were prepared using Cremophor RH40 as surfactant and PG (Propylene glycol) and PEG (Polyethylene Glycol 400) as the structurants. A concentrate mix was prepared by adding all surfactants and co-surfactants and mixing following each addition. PEG 4000 or 8000 were added and heated to melt. The required amount of tributyrin was then added to the mixture. The required amount of CBD and/or THC was added. The concentrate was then homogenized using magnetic stirring at 1500 rpm till solidification. Tributyrin based SEDDS concentrates were prepared with Cremophor RH40: PEG400: propylene glycol (350:350:350) and 200 part of tributyrin as shown in Table 13. Similar SEDDS were also prepared by substituting Cremophor RH40 with Gelucire and TPGS as surfactants.

TABLE 13
Compositions of various blank and drug-loaded liquid and semi-solid SEDDS
concentrates at water dilution 1:40

| | | | | | | PEG | PEG | | | SEDDS | | | | |
|---|---|---|---|---|---|---|---|---|---|---|---|---|---|---|
| | TBN | GLC | CRM | TPGS | PG | 400 | 4000 | CBD | THC | (1:40) | | Size | | |
| | (mg) | (mg) | (mg) | (mg) | (mg) | (mg) | (mg) | (mg) | (mg) | Appearanc | Consistency | (nm) | Distribution | PDI |
| TB1 | 200 | 0 | 350 | 0 | 350 | 350 | 0 | 65 | 3.25 | Milky | Low | 94.98 | P1 = 126.9 (100%) | 0.258 |
| TB2 | 200 | 0 | 350 | 0 | 350 | 260 | 90 | 65 | 3.25 | Milky | Good | 94.17 | P1 = 134.8 (96.5%) | 0.385 |
| | | | | | | | | | | | | | P2 = 4410(3.5%) | |
| TB3 | 200 | 350 | 0 | 0 | 350 | 350 | 0 | 65 | 3.25 | Milky | Low | 291.2 | P1 = 180.4 (49.7%) | 0.643 |
| | | | | | | | | | | | | | P2 = 687.3 (43.2%) | |
| | | | | | | | | | | | | | P3 = 5078 (7.1%) | |
| TB4 | 200 | 350 | 0 | 0 | 350 | 260 | 90 | 0 | 0 | Milky | Good | 202.6 | P1 = 297.1 (97.3%) | 0.373 |
| | | | | | | | | | | | | | P2 = 4935 (2.7%) | |
| TB5 | 200 | 350 | 0 | 0 | 350 | 260 | 90 | 65 | 3.25 | Milky | Good | 320.4 | P1 = 806.9 (49.2%) | 0.642 |
| | | | | | | | | | | | | | P2 = 145.2 (47.0%) | |
| | | | | | | | | | | | | | P3 = 5389 (3.8%) | |
| TB6 | 200 | 0 | 0 | 350 | 350 | 350 | 0 | 65 | 3.25 | Milky | Low | 94.44 | P1 = 140.8 (98.4%) | 0.390 |
| | | | | | | | | | | | | | P2 = 4779 (1.6%) | |
| TB7 | 200 | 0 | 0 | 350 | 350 | 260 | 90 | 0 | 0 | Clear | Good | 32.37 | P1 = 20.35 (56.2%) | 0.694 |
| | | | | | | | | | | | | | P2 = 992.8 (43.8%) | |
| TB8 | 200 | 0 | 0 | 350 | 350 | 260 | 90 | 65 | 3.25 | Milky | Good | 102.7 | P1 = 145.0 (97.5%) | 0.268 |
| | | | | | | | | | | | | | P2 = 18.27 (2.5%) | |

TBN: tributyrin, GLC: gelucire, CRM: Cremophor RH40, TPGS: d-α-tocopheryl polyethylene glycol 1000 succinate, PG: propylene glycol

Effect of PEG4000 on Gel to Sol Consistency

PEG 4000 has been used as the solidifying agent to get sol to gel consistency. Formulations with Cremophor RH40: PEG400: Propylene glycol (350:350:350) and 200 part of tributyrin were prepared initially using 90 mg (6.9% w/v) of PEG4000. The amount of PEG4000 was adjusted by reducing the amount of PEG400 from 350 to 260 mg in the stock (26.55% to 19.72% w/w). Similar formulations were prepared using Gelucire or TPGS as the surfactant in place of Cremophor RH40 (see Table 13). The formulations were prepared with and without CBD (50 mg/ml in final formulation or 5% w/v). The prepared concentrates showed good SEDDS formation and good average droplet size and PDI on dilution at 1:40 with Distilled Water. TPGS based SEDDS concentrate also showed good results however the Gelucire based mixtures showed higher size and PDI.

Effect of Increasing Concentration of PEG4000

The effect of increasing the concentration of PEG4000 and its compatibility with gelatin capsule was assessed. Different amounts of PEG 4000-90 mg (6.92%), 120 mg (9.25%) and 150 mg (11.5%) —were used in formulation containing Cremophor RH40 as surfactant with only 50 mg/ml (5% of total formulation) CBD as drug. The consistency was found to increase proportionately (visual observation) and was found to be better with PEG4000 content of 120 mg and 150 mg. The increase in PEG4000 concentration did not seem to have significant impact on the size distribution of the reconstituted SEDDS and was found to be more or less similar in TB10 (90 mg PEG4000), TB12 (120 mg PEG4000) and TB14 (150 mg PEG4000). The results are shown in Table 14-1.

Further SEDDS were prepared with PEG4000 (150 mg) using the different surfactants Cremophor RH40, Gelucire and TPGS as shown in Table 14-2. The size distribution of the drug loaded formulations on reconstitution at 1:40 were found to be satisfactory for formulations with Cremophor RH40 (TB14) and TPGS (TB18) as surfactant. However, the size and PDI was slightly higher for Gelucire (TB16).

TABLE 14-1
Effect of increasing PEG4000 conccentration on physical properties of
tributyrin SEDDS cpmcemtrates base on Cremophor RH40

| | | | | | PEG | PEG | | SEDDS | | | | | Z- |
|---|---|---|---|---|---|---|---|---|---|---|---|---|---|
| | TBN | CRM | CRM | PG | 400 | 4000 | CBD | (1:40) | | Size | | | potential |
| | (mg) | (mg) | (mg) | (mg) | (mg) | (mg) | (mg) | Appearanc | Consistency | (nm) | Distribution | PDI | (-mv) |
| TB9 | 200 | 350 | 350 | 350 | 260 | 90 | 0 | Milky | Good | 129.7 | P1 = 346.4 (64.5%) | 0.488 | — |
| | | | | | | | | | | | P2 = 23.57 (23%) | | |
| | | | | | | | | | | | P3 = 3826 (12.5%) | | |
| TB10 | 200 | 350 | 350 | 350 | 260 | 90 | 65 | Milky | Good | 146.8 | P1 = 209.2 (92.1%) | 0.376 | 28.7 |
| | | | | | | | | | | | P2 = 3938 (6.0%) | | |
| | | | | | | | | | | | P3 = 21.43 (1.9%) | | |
| TB11 | 200 | 350 | 0 | 320 | 260 | 120 | 0 | Milky | Good | 111.9 | P1 = 584 (65.1%) | 0.376 | — |
| | | | | | | | | | | | P2 = 27.0 (26.2%) | | |
| | | | | | | | | | | | P3 = 4196 (8.7%) | | |
| TB12 | 200 | 350 | 0 | 320 | 260 | 120 | 65 | Milky | Good | 171.4 | P1 = 205.1 (96.4%) | 0.376 | 24.9 |
| | | | | | | | | | | | P2 = 5184 (3.6%) | | |
| TB13 | 200 | 350 | 0 | 290 | 260 | 150 | 0 | Milky | Good | 149.0 | P1 = 286.9 (61.1%) | 0.303 | — |
| | | | | | | | | | | | P2 = 24.88 (26.4%) | | |
| | | | | | | | | | | | P3 = 4177 (12.5%) | | |
| TB14 | 200 | 350 | 0 | 290 | 260 | 150 | 65 | Milky | Good | 165.9 | P1 = 235.5 (95.3%) | 0.396 | 32.0 |
| | | | | | | | | | | | P2 = 4867 (3.1%) | | |
| | | | | | | | | | | | P3 = 19.89 (1.5% | | |

TBN: tributyrin, CRM: Cremophor RH40, PG: propylene glycol

TABLE 14-2
Effect of surfactant type on the physical properties of bland and CBD-
loaded tributyrin-based semi-solid SEDDS concentrates, prepared with 150 mg of
PEG4000

| | | | | | | PEG | PEG | | SEDDS | | | | | |
|---|---|---|---|---|---|---|---|---|---|---|---|---|---|---|
| | TBN | GLC | CRM | TPGS | PG | 400 | 4000 | CBD | (1:40) | | Size (nm) | | CBD content | |
| | (mg) | (mg) | (mg) | (mg) | (mg) | (mg) | (mg) | (mg) | Appearanc | Consistency | [PDI] | Distribution | Day 0 | Day 10 |
| TB13 | 200 | 0 | 350 | 0 | 290 | 260 | 150 | 0 | Milky | Good | 149.0 | P1 = 286.9 (61.1%) | — | — |
| | | | | | | | | | | | [0.303] | P2 = 24.88 (26.4%) | | |
| | | | | | | | | | | | | P3 = 4177 (12.5%) | | |
| TB14 | 200 | 0 | 350 | 0 | 290 | 260 | 150 | 65 | Milky | Good | 165.9 | P1 = 235.5 (95.3%) | 92.23 | 92.05 |
| | | | | | | | | | | | [0.396] | P2 = 4867 (3.1%) | | |
| | | | | | | | | | | | | P3 = 19.89 (1.5%) | | |
| TB15 | 200 | 350 | 0 | 0 | 290 | 260 | 150 | 0 | Milky | Good | 211.1 | P1 = 257.5 (98.0%) | — | — |
| | | | | | | | | | | | [0.314] | P2 = 5182 (2.0%) | | |
| TB16 | 200 | 350 | 0 | 0 | 290 | 260 | 150 | 65 | Milky | Good | 287.0 | P1 = 369.4 (74.3) | 96.47 | 94.14 |
| | | | | | | | | | | | [0.502] | P3 = 5560 (1.2%) | | |
| TB17 | 200 | 0 | 0 | 350 | 290 | 260 | 150 | 0 | Milky | Good | 48.58 | P1 = 24.32 (50.7%) | — | — |
| | | | | | | | | | | | [0.551] | P2 = 587.0 (46.3%) | | |
| | | | | | | | | | | | | P3 = 4070 (2.9%) | | |
| TB18 | 200 | 0 | 0 | 350 | 290 | 260 | 150 | 65 | Milky | Good | 150.0 | P1 = 225.1 (95.27%) | 94.52 | 95.56 |
| | | | | | | | | | | | [0.370] | P2 = 4207 (3.2%) | | |
| | | | | | | | | | | | | P3 = 22.18 (1.0%) | | |

TBN: tributyrin, GLC: gelucire, CRM: Cremophor RH40, TPGS: d-α-tocopheryl polyethylene glycol 1000 succinate, PG: propylene glycol

Construction of Pseudo-Ternary Phase Diagrams

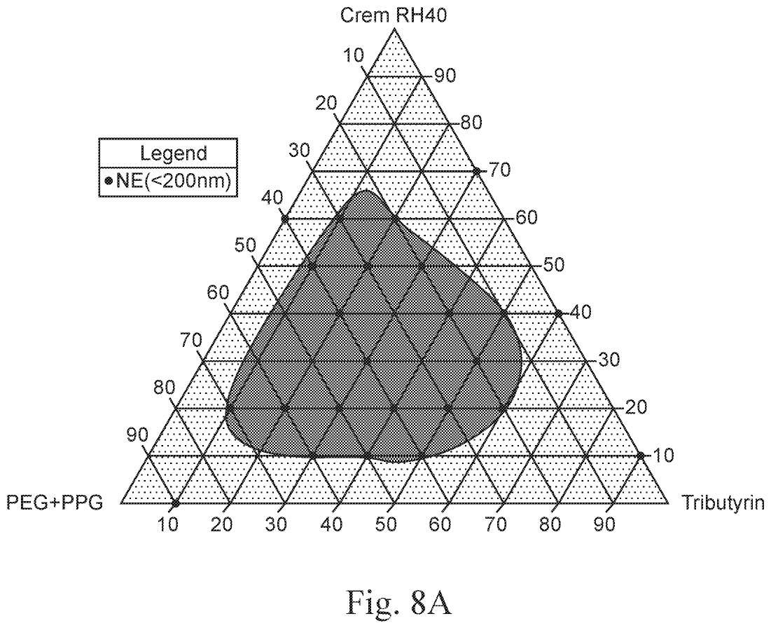

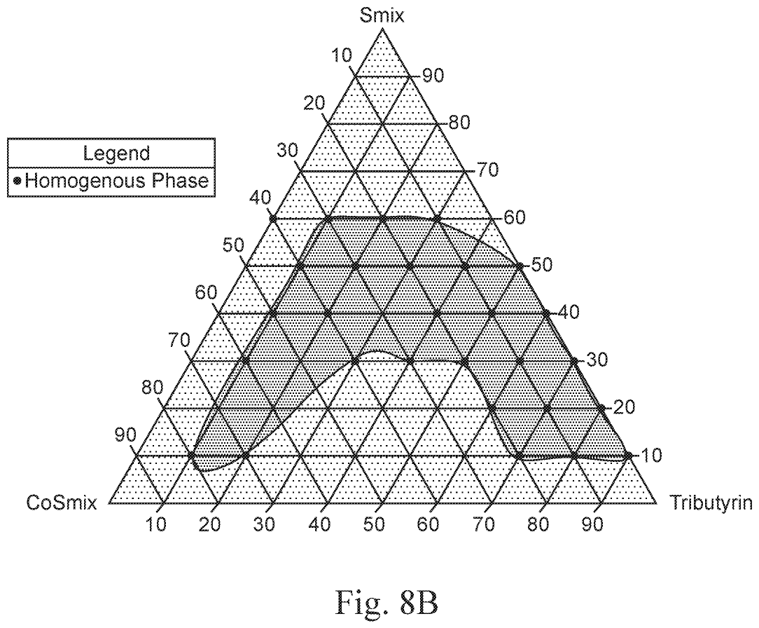

Ternary diagrams with different concentrations of surfactant, co-surfactants mixture and tributyrin were plotted, each representing a corner of the triangle. Various combinations were prepared by varying the content of Cremophor RH40 (Surfactant) 10-60%, Tributyrin (10-90%) and mix of PEG400 and Propylene glycol in 1:1 ratio (0-100%). A total of 46 combinations were prepared and evaluated for nanoemulsification ability and physical stability on overnight storage (SEDDS formation, droplet size and PDI). The combinations which formed submicron emulsions with size <200 nm and which those showed separation of phases on overnight storage were plotted as pseudo ternary diagram (FIG. 8A).

It was observed that some combinations within this nanoemulsion region showed separation on overnight standing. A region of tributyrin (30-60%), Cremophor RH40 (10-30%) and PEG+PPG (20-70%) showed separation of phases. Hence a combination of tributyrin 16%, Cremophor RH40 28% and PEG+PPG mix 56% was selected from the nanoemulsion region considering the physically stable region as shown in FIG. 8B.

Thermo-Reversibility and Reconstitution at 37° C.

To assess the behavior of reconstituted SEDDS in body temperature, SEDDS reconstitution was carried out at 37° C. and the thermo-reversibility was observed visually for TB10, TB12 and TB14. To 2 ml of distilled water maintained at 37° C., about 50 mg of the semi-solid formulations were added and stirred at 200 rpm at 37° C. for 1 hour. The average size and PDI of the reconstituted SEDDS were found to be satisfactory in all the three tested formulations. The thermo-reversibility was found to be within 45-60 min for TB10 (90 mg PEG4000) and TB12 (120 mg PEG4000), and 45-75 min for TB14 (150 mg PEG4000). The results are shown in Table 15-1. The CBD content in the various semi-solid SEDDS was close to 93% for all the formulations. The relatively low content was attributed to the lack of efficiency of the extraction in the semi solid formulations.

TABLE 15-1
Effect of increasing concentrations of PEG4000 in tributyrin semi-solid
SEDDS, reconstituted at 37° C. under 200 rpm stirring for 1 hour, dilution 1:40

| | | | | PEG | PEG | | | Thermo- | | | CBD in |
|---|---|---|---|---|---|---|---|---|---|---|---|
| | TBN | CRM | PG | 400 | 4000 | CBD | SEDDS | reversab. | Size (nm) | | conc. |
| | (mg) | (mg) | (mg) | (mg) | (mg) | (mg) | appearance | at 37° C. | [PDI] | Distribution | (%) |
| TB10 | 200 | 350 | 350 | 260 | 90 | 65 | Milky | 45-60 min | 213.5 | P1 = 273.2 (96.6%) | 92.54 |
| | | | | | | | | | [0.442] | P2 = 5215 (3.4%) | |
| TB12 | 200 | 350 | 320 | 260 | 120 | 65 | Milky | 45-60 min | 216.7 | P1 = 273.0 (93.2%) | 93.26 |
| | | | | | | | | | [0.399] | P2 = 4661 (6.8%) | |
| TB14 | 200 | 350 | 290 | 260 | 150 | 65 | Milky | 45-75 min | 232.5 | P1 = 275.4 (96.5%) | 93.23 |

TBN: tributyrin, CRM: Cremophor RH40, PG: propylene glycol

Additional modifications of the extraction procedure improved the drug content. In the reconstitution of formulation based on 150 mg of PEG4000 using different surfactants, the results were found to be similar. The size distribution was found to be satisfactory for TB14 and TB18 and showed higher PDI and larger (17-18%) particles for TB16. The reconstitution behavior was repeated after 10 days of the same formulations and was found to have no significant difference. The thermo-reversibility of the formulations at 37° C. was found to be within 45-75 min. The results are shown in Table 15-2.

TABLE 15-2
Effect of surfactant type on tributyrin semi-solid SEDDS, 150 mg
PEG4000, reconstituted at 37° C. under 200 rpm stirring for 1 hour, dilution 1:40

| | | | | | | PEG | PEG | | Thermo- | Day 0 | | Day 10 | |
|---|---|---|---|---|---|---|---|---|---|---|---|---|---|
| | TBN | GLC | CRM | TPGS | PG | 400 | 4000 | CBD | reversab. at | Size (nm) | | Size (nm) | |
| | (mg) | (mg) | (mg) | (mg) | (mg) | (mg) | (mg) | (mg) | 37° C. | [PDI] | Distribution | [PDI] | Distribution |
| TB14 | 200 | 0 | 350 | 0 | 290 | 260 | 150 | 65 | 45-75 min | 232.5 | P1 = 275.4 (96.5%) | 229.9 | P1 = 337.5 (94.2%) |
| | | | | | | | | | | [0.410] | P2 = 5271 (3.5%) | [0.664] | P2 = 5157 (5.8%) |
| TB16 | 200 | 350 | 0 | 0 | 290 | 260 | 150 | 65 | 45-60 min | 213.9 | P1 = 206.7 (82.7%) | 361.9 | P1 = 283.0 (979%) |
| | | | | | | | | | | [0.358] | P2 = 1155 (10.8%) | [0.426] | P2 = 5560 (2.1 %) |
| | | | | | | | | | | | P3 = 4689 (6.5%) | | |
| TB18 | 200 | 0 | 0 | 350 | 290 | 260 | 150 | 65 | 45-75 min | 202.6 | P1 = 263.0 (96.4%) | 168.9 | P1 = 241.2 (88.9%) |
| | | | | | | | | | | [0.454] | P2 = 5247(3.6%) | [0.405] | P2 = 3170 (9.4%) |
| | | | | | | | | | | | | | P3 = 18.57 (1.7%) |

TBN tributynn, GLC: gelucire, CRM: Cremophor RH40, TPGS: d-α-tocopheryl polyethylene glycol 1000 succinate, PG: propylene glycol

Effect of Increasing the Drug Concentration

The effect of increasing drug concentration was observed by increasing the drug concentration from 50 mg/ml to 100 mg/ml as can be seen in Table 16. The effect of this variation on the ability for SEDDS formation and on the average droplet size and size distribution of the formulation on dilution with water at 1:40 dilution and upon reconstitution at 37° C. were observed. It is noted that there is no marked difference in the particle size distribution either following reconstitution at 37° C. over 1 hour.

Physical Stability of the SEDDS Concentrate [Liquid] after Reconstitution at Different Conditions

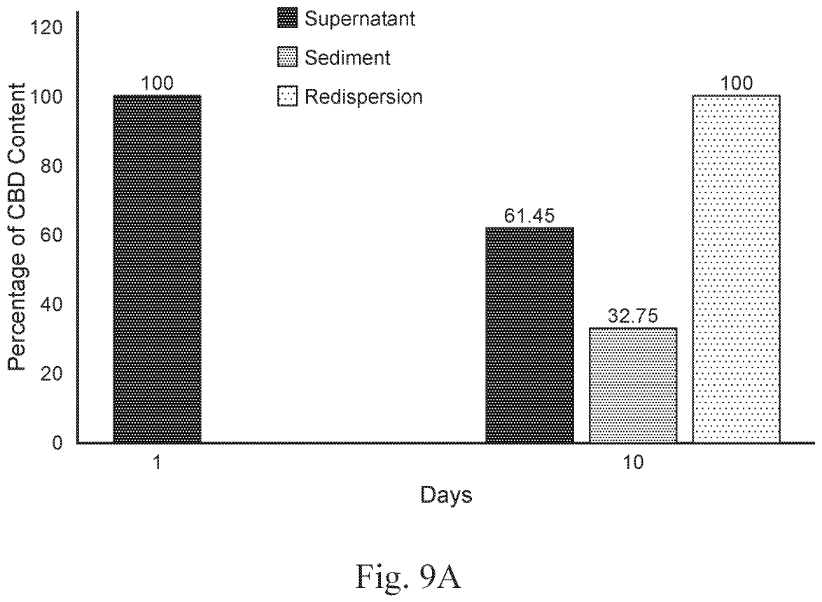

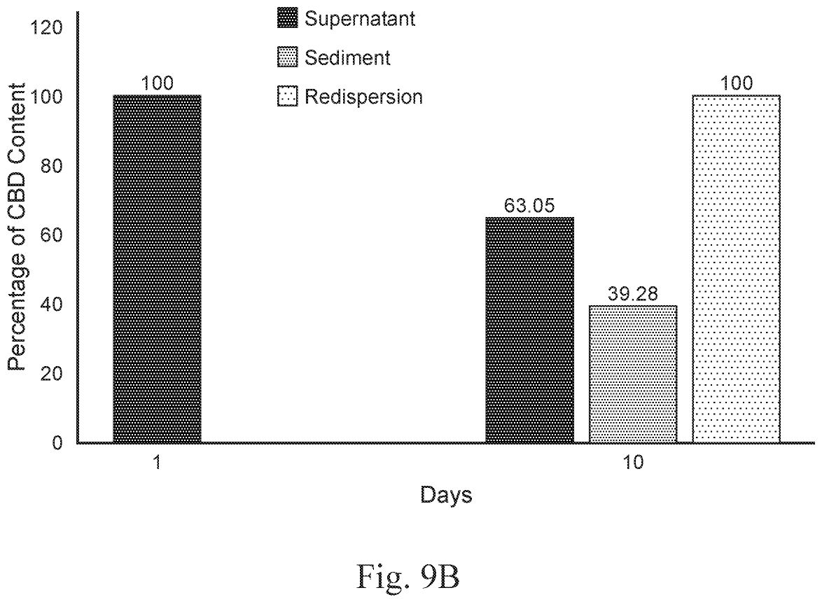

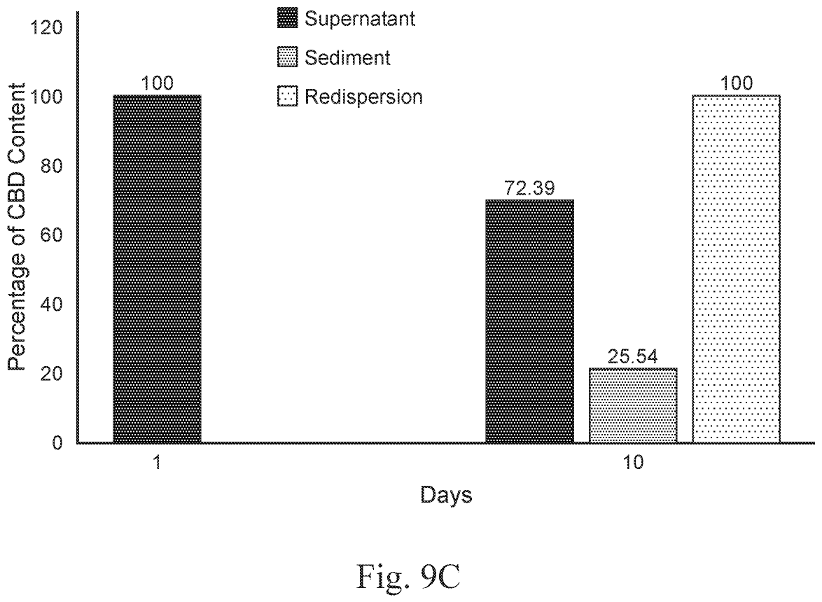

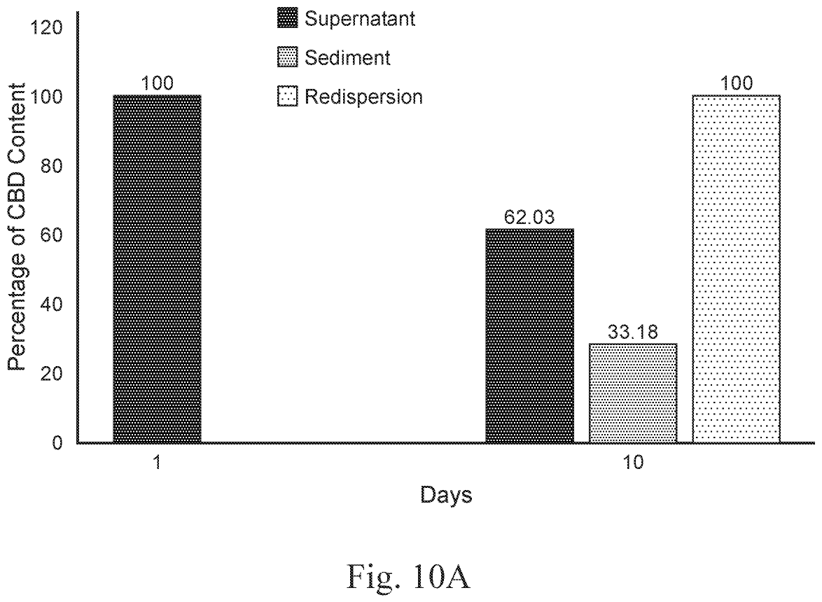

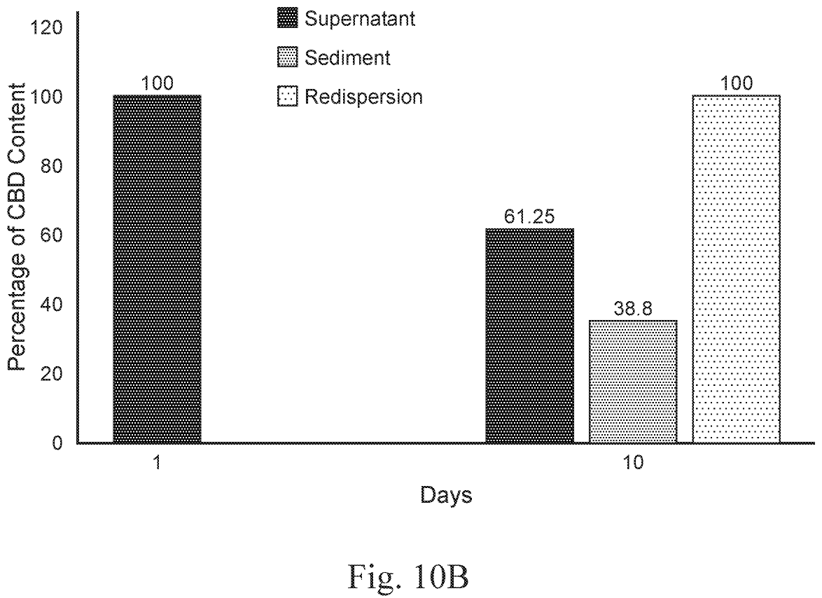

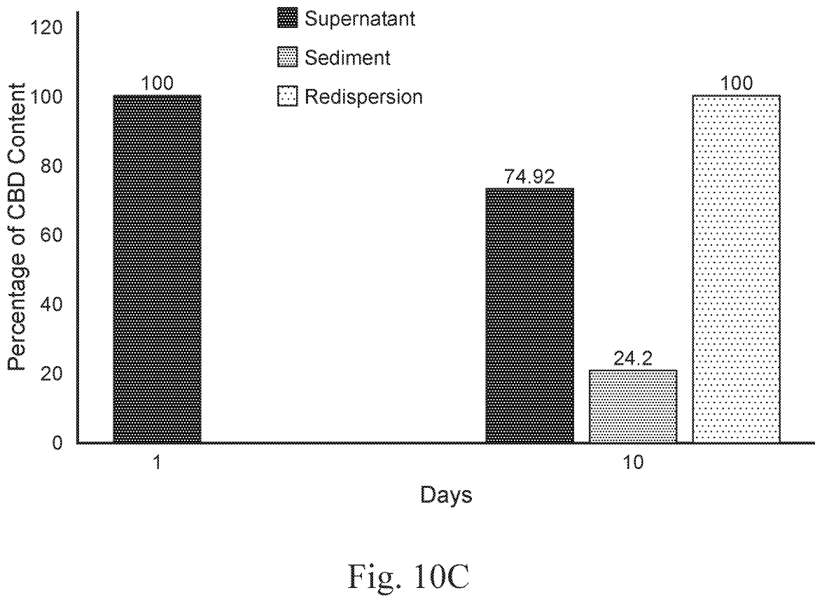

Physical stability of the liquid SEDDS concentrate (TB1) at 1:10, 1:20 and 1:40 dilutions was observed. Following 10 days, the amount of drug in the supernatant, sediment and redispersion was determined. The amount of sedimentation was found to be higher in 1:10 and 1:20 dilutions showing drug content of 61.45% and 63.05% in the supernatant respectively. The drug content of supernatant at 1:40 dilution showed 72.39% at room temperature. The results were found to be similar at 4° C. In all tested dilutions, redispersion showed 100% drug content. The results are presented in FIGS. 9A-10C. These results indicate that the formulations are of limited physically stability over time when diluted although CBD remain intact in the various separated phases.

Use of PEG8000 as Solidifying Agent

PEG8000 has been used in the formulations to increase the physical stability of the filled capsules on storage. Formulations with PEG4000:PEG8000 at 75 mg:75 mg were prepared and tested for consistency and tendency to form SEDDS as shown in Table 17.

TABLE 16
Effect of increased drug loading in tributyrin semi-solid SEDDS

| | | | | PEG | PEG | | | SEDDS | Thermo- | | | CBD in |
|---|---|---|---|---|---|---|---|---|---|---|---|---|
| | TBN | CRM | PG | 400 | 4000 | CBD | THC | (1:40) | reversab. | Size (nm) | | conc. |
| | (mg) | (mg) | (mg) | (mg) | (mg) | (mg) | (mg) | appearance | at 37° C. | [PDI] | Distribution | (%) |
| TB19 | 200 | 350 | 290 | 260 | 150 | 130 | 6.5 | Milky | 30-45 min | 202.8 | P1 = 242.9 (95.9%) | 87.54 |
| | | | | | | | | | | [0.350] | P2 = 5015 (4.1%) | |
| TB19** | 200 | 350 | 290 | 260 | 150 | 65 | 6.5 | — | 30-45 min | 252.1 | P1 = 234.3 (86.9%) | — |
| | | | | | | | | | | [0.474] | P2 = 45.29 (7.4%) | |
| | | | | | | | | | | | P3 = 5370 (5.8%) | |

TBN: tributyrin, CRM: Cremophor RH40, PG: propylene glycol
**reconstituted at 37° C. under 200 rpm stirring for 1 hour

TABLE 17
Effect of high molecular weight mixture of PEG in tributyrin semi-solid
SEDDS, dilution 1:10

| | | | | PEG | PEG | PEG | | | |
|---|---|---|---|---|---|---|---|---|---|
| | TBN | CRM | PG | 400 | 4000 | 8000 | CBD | Size (nm) | |
| | (mg) | (mg) | (mg) | (mg) | (mg) | (mg) | (mg) | [PDI] | Distribution |
| TB20 | 200 | 350 | 290 | 260 | 75 | 75 | 0 | 267.9 | P1 = 455.8 (61.2%) |
| | | | | | | | | [0.387] | P2 = 3804 (19.5%) |
| | | | | | | | | | P3 = 23.13 (19.3%) |
| TB21 | 200 | 350 | 290 | 260 | 75 | 75 | 65 | 191.1 | P1 = 270.4 (93.5%) |
| | | | | | | | | [0.398] | P2 = 4515 (5.1%) |
| | | | | | | | | | P3 = 26.08 (1.4%) |
| TB22 | 200 | 350 | 290 | 260 | 75 | 75 | 130 | 200.5 | P1 = 254.2 (93.7%) |
| | | | | | | | | [0.393] | P2 = 4860 (6.3%) |

TBN: tributyrin, CRM: Cremophor RH40, PG: propylene glycol

Physical Stability of Capsules on Storage in Room Condition and 4° C. and Effect of the Formulations Containing PEG 4000: PEG8000 (75 mg: 75 mg).

The compatibility of the formulations with the capsule shell and the consistency of the formulations on storage was observed as explained above. The optimized new formulations (blank) TB 20 and TB21 (loaded with drug) were filled into gelatin capsules (0.6 ml in Size 0 and 0.25 ml in Size 3). Two different types of Size 0 capsules were used (BOL Pharma and Theo200). These capsules were then stored under Room Temperature (25° C. at 60% humidity controlled condition) (5 capsules each). The results have been shown in Table 18.

TABLE 18
physical stability of hard capsules on horizontally positioned storage at room temperature and 4° C.

| | | Room temperature | | 4° C. | |
|---|---|---|---|---|---|
| | Day | Consistency | Capsule shell compatibility | Consistency | Capsule shell compatibility |
| TB20 | 7 | Good | Good | Good | Good |
| | 14 | Good | Good | Good | Good |
| | 21 | Good | Good | Good | Good |
| | 28 | Good | Source 1: swelling observed | Good | Good |
| | | | in size 0, size 3 intact | | |
| | | | source 2: intact | | |
| | 45 | Good | Source 1: swelling and | Good | Good |
| | | | leakage in size 0 and 3 | | |
| | | | source 2: intact | | |
| TB21 | 7 | Good | Good | Good | Good |
| | 14 | Good | Good | Good | Good |
| | 21 | Good | Good | Good | Good |
| | 28 | Good | Good | Good | Good |
| | 45 | Good | Source 1 and 2: leakage in | Good | Source 1: leakage in size 0 and 3 |
| | | | some size 0 capsules, size 3 | | source 2: size 0 intact |
| | | | remained intact | | |

The gelatin Size 3 capsules (source 1) were found to be stable for one month but started showing swelling after 45 days at room condition. The gelatin Size 0 capsules (source 1) remained stable for 21 days and started showing swelling after one month. The gelatin capsules (source 2) remained stable for more than 60 days with no sign of any deformation. The capsules at 4° C. remained stable for one month however swelling and leakage was observed in drug loaded Gelatin Size 0 and size 3 capsules (source 1) after 45 days while the Size 0 (source 2) and blank remained stable for 2 months.

Reconstitution at 37° C.

The thermo-reversibility and reconstitution of the formulation TB21 was observed at 37° C. The thermo-reversibility of the formulation was found to be satisfactory with the semi-solid SEDDS liquefying within 30-45 min. The size and PDI of the formulation on reconstitution at 37° C. in 1:40 ratio and on stirring at 200 rpm for 1 hour also showed no significant difference, as seen in Table 19.

TABLE 19
Tributyrin semi-solid SEDDS, reconstituted at 37° C. at 200 rpm stirring for 1 hour

| | | | | PEG | PEG | PEG | | | Thermo- | | |
|---|---|---|---|---|---|---|---|---|---|---|---|
| | TBN | CRM | PG | 400 | 4000 | 4000 | CBD | THC | reversab. | Size (nm) | |
| | (mg) | (mg) | (mg) | (mg) | (mg) | (mg) | (mg) | (mg) | at 37° C. | [PDI] | Distribution |
| TB21 | 200 | 350 | 290 | 260 | 75 | 75 | 130 | 6.5 | 30-45 min | 243.63 | P1 = 304.0 (81.9%) |
| | | | | | | | | | | [0.365] | P2 = 88.84 (18.1%) |

Pharmacokinetic (PK) Studies for CBD- and THC-Loaded SEDDS Based on Tributyrin

Study Protocol

The PK studies were carried out on rats. Three animals were used in each time point. At each test point, animals were sacrificed and blood (5-6 ml) withdrawn from the heart and collected in heparinized tubes.

Each rat was orally administered with 1 mil of the test formulation. Blood samples were withdrawn at different time points: 0, 0.5, 1, 2, 4, 6, 10 and 24 hours. The blood samples were centrifuged at 4000 rpm and the plasma was separated and collected in clean polyethylene tubes and kept at −80° C. until analysis using LC-MS/MS.

TB1 liquid SEDDS formulations were tested: 200 parts tributyrin, 350 parts Cremophor RH40, 350 parts PEG400, 350 parts PG. CBD and THC were added according to the required ratio (20:1, 1:1 or 1:5). Only the formulation with CBD:THC ratio of 20:1 was compared to the respective semi-solid SEDDS.

TB2 semi-solid SEDDS formulations were tested as well: 15.17 wt % tributyrin, 26.55 wt % Cremophor RH40, 22 wt % PG, 19.72 wt % PEG400, 11.38 wt % PEG4000, 5 wt % CBD and 0.25 wt % THC (CBD:THC ratio of 20:1). Similar formulations were prepared with 0.5 wt % CBD and 0.5 wt % THC (1:1 ratio), and 0.5 wt % CBD and 2.5 wt % THC (1:5 ratio).

Determination of CBD and THC by LC-MS

Method

Cannabidiol (CBD) and Tetrahydrocannabinol (THC) were supplied by BOLPHARMA (Rivadim, Israel). Quantitative Analysis of cannabinoids was carried out by using the ISQ™ EC Single Quadrupole Mass Spectrometer of CBD and THC.

Instrumentation

The HPLC system consisted of a Dionex/Thermo Scientific UltiMate 3000 UHPLC systems fitted with a 100 μL sample loop, Dionex™ UltiMate™ 3000 Rapid Separation Diode Array Detector, and integration software Chromeleon Chromatography Data System (CDS).

Chromatographic separation was performed with guard column Luna® 5 μm C₁₈(2) 100 Å, LC Column 150×4.6 mm, using an isocratic mobile phase of water (0.1% formic acid): acetonitrile (0.1% formic acid) 10:90 at a flow rate of 1 mL/min for 10 in minisocratic condition. The column temperature was 40° C. and the injection volume was 100 μL.

LCMS-ISQ™ EC Single Quadrupole MS Analysis

Dual ion electrospray ionization (DUIS) in positive was used for ionization of the analytes on the ISQ™ EC. A positive selected ion monitoring (SIM) modes were used simultaneously for analysis. Details of the MS parameters are shown: CBD MS (MS/z): 314-350 retention time 5.3 minutes; THC MS (MS/z); 314-350 retention time 9.8 minutes; Nebulizing gas flow 20 L/min; Drying gas flow 205 L/min; DL Temperature 350° C. and Heat Block Temperature 500° C.

Extraction Method

Two ml (milliliters) of plasma samples were transferred two 2 new 10 ml tubes (each contained 1 ml). To each tube, 4 ml acetonitrile (CAN) were added and vortexed 3 times over 30 min. The samples were re-centrifuged (4000 rpm, 10 min). Then, the supernatant was evaporated under nitrogen flow at 55° C. After evaporation, 500 μL of ACN:H₂O HPLC, 80:20% were added to each tube, vortexed 3 times over 30 min and then transferred to the second tube. Following again vortex, the samples were centrifuged (4000 rpm, 5 min) and 350 μL of the supernatant were transferred to a vial for MS evaluation.

Calibration curves were prepared in plasma and ACN, separately. Concentrations of CBD and THC were 0-500 ng/mL

Results

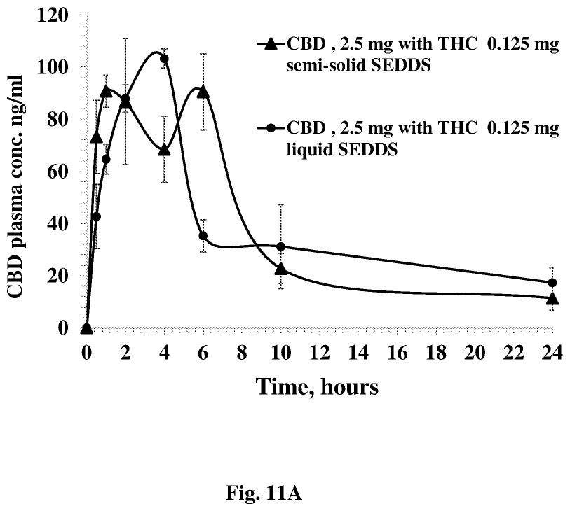

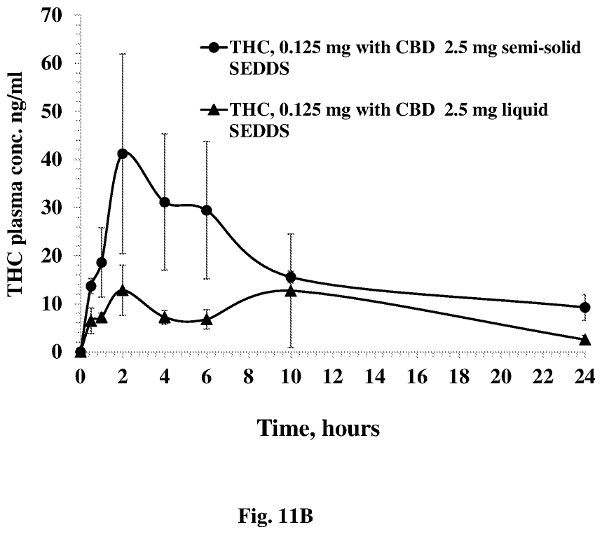

The mean pharmacokinetic profiles of the CBD and THC at a fixed doses of 2.5 mg and 0.125 mg per rat, respectively are presented in FIGS. 11A-11B and the values of the various PK parameters are reported in Table 20. It was observed that there was no significant difference in the profiles of CBD between the liquid and semi-solid SEDDS formulations, whereas for the THC profile—there was an increase in bioavailability for the semi-solid SEDDS compared to the liquid SEDDS (2.45 folds).

TABLE 20
Pharmacokinetics parameters of CBD and THC following oral
administration of liquid and semi-solid SEDDS, CBD:THC ratio of 20:1

| | 2.5 mgCBD/ | | 0.125 mgTHC/ | |
|---|---|---|---|---|
| | 0.125 mg THC | | 2.5 mg CBD | |
| Parameter | semi-solid | Liquid | semi-solid | Liquid |
| T_{1/2} (hours) | 7.17 | 17.3 | 10.48 | 11.27 |
| Tₘₐₓ (hours) | 1.00 | 4.00 | 2.00 | 2.00 |
| Cₘₐₓ (ng/ml) | 90.74 | 103.29 | 41.17 | 12.81 |
| Clast_obs/Cₘₐₓ | 0.12 | 0.17 | 0.22 | 0.20 |
| AUC 0-inf_obs | 1044.88 | 1350.36 | 576.52 | 235.38 |
| (ng/ml × h) | | | | |
| MRT 0-inf_obs | 10.05 | 21.30 | 16.15 | 14.63 |
| (hours) | | | | |
| Vz/F_obs | 74225.73 | 138757.59 | 9836.58 | 25906.19 |
| (ml/kg) | | | | |
| Cl/F_obs | 7177.82 | 5554.06 | 650.46 | 1593.18 |
| (ml/kg × h) | | | | |

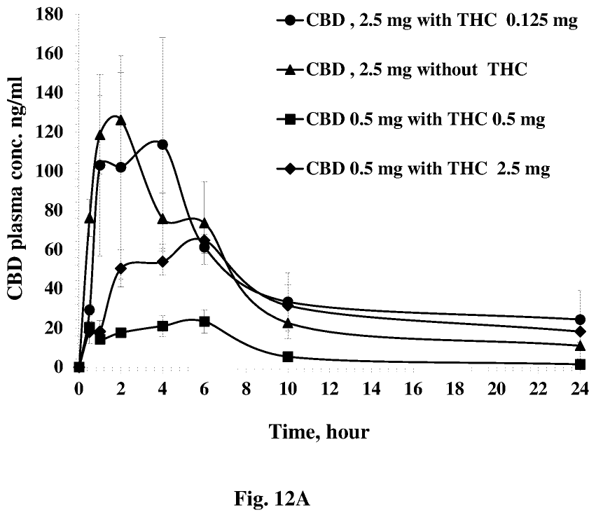

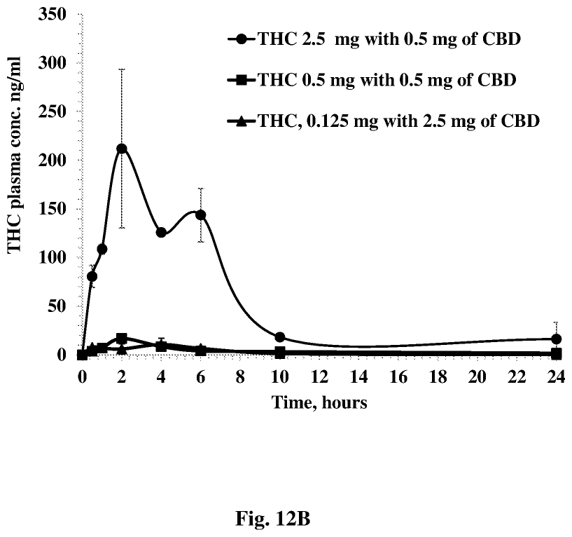

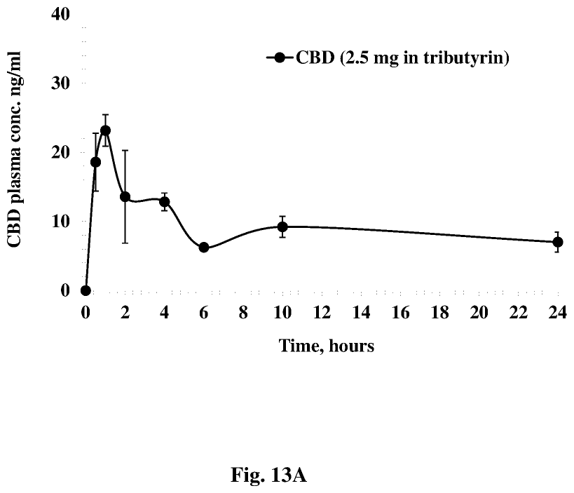

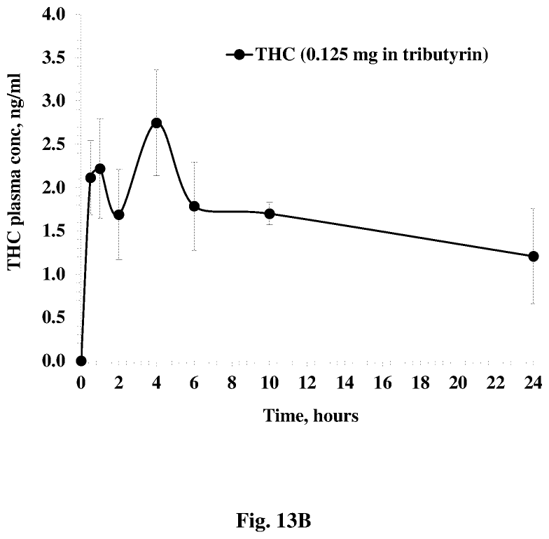

Results for various ratios of THC and CBD in different semi-solid SEDDS formulations are shown in FIGS. 12A-12B, and in Table 21-1. Results for reference examples of CBD and THC in tributyrin are shown in FIGS. 13A-13B and Table 21-2.

The bioavailability (AUC) of CBD alone, and combined with THC (20:1 CBD:THC) as compared to tributyrin alone are 7.15 and 10 folds higher, respectively. The CBD bioavailability (AUC) increased markedly when THC concentration increased form 1:1 to 1:5; the THC bioavailability (AUC) increased markedly with the increase in THC dos irrespective of the dose of CBD. The bioavailability of THC value increased by 100% compared to the bioavailability of THC in tributyrin as well (at a ratio of CBD:THC of 20:1).

TABLE 21-1
PK parameters for CBD and THC in different semi-solid formulations, at
various CBD:THC ratios following oral administration in rats

| | CBD formulations | | | | THC formulations | | |
|---|---|---|---|---|---|---|---|
| | [CBD mg/THC mg] | | | | [THC mg/CBD mg] | | |
| parameter | 2.5/0 | 2.5/0.125 | 0.5/0.5 | 0.5/2.5 | 2.5/0.5 | 0.5/0.5 | 0.125/2.5 |
| T1/2 (hours) | 6.44 | 10.73 | 4.95 | 11.08 | 5.94 | 14.71 | 4.07 |
| Tmax (hours) | 2.00 | 4.00 | 6.00 | 6.00 | 2.00 | 2.00 | 4.00 |
| Cmax (ng/ml) | 126.12 | 113.54 | 23.27 | 64.71 | 211.93 | 16.96 | 10.74 |
| Clast_obs/Cmax | 0.09 | 0.21 | 0.06 | 0.28 | 0.08 | 0.11 | 0.03 |
| AUC 0-inf_obs | 1068.06 | 1500.14 | 226.17 | 1098.27 | 1540.42 | 143.03 | 77.27 |
| (ng/ml × h) | | | | | | | |
| MRT 0-inf_obs | 9.02 | 16.19 | 7.14 | 17.48 | 8.60 | 17.58 | 6.09 |
| (hours) | | | | | | | |
| Vz/F_obs (ml/kg) | 65199.12 | 77413.04 | 47354.57 | 21827.03 | 41728.99 | 222587.54 | 28469.79 |
| Cl/F_obs | | | | | | | |
| (ml/kg × h) | 7022.07 | 4999.52 | 6632.19 | 1365.79 | 4868.80 | 10487.40 | 4853.36 |

TABLE 21-2
Pharmacokinetics parameters of CBD and THC in tributyrin as a carrier
following oral administration, CBD:THC ratio of 20:1

| | 2.5 mgCBD/ | 0.125 mgTHC/ |
|---|---|---|
| Parameter | 0.125 mg THC | 2.5 mg CBD |
| T_{1/2} (hours) | 51.99 | 30.87 |
| Tₘₐₓ (hours) | 2.00 | 4.00 |
| Cₘₐₓ (ng/ml) | 6.71 | 2.7 |
| Clast_obs/Cₘₐₓ | 0.21 | 0.44 |
| AUC 0-inf_obs (ng/ml × h) | 149.39 | 39.81 |
| MRT 0-inf obs (hours) | 74.65 | 43.82 |
| Vz/F_obs (ml/kg) | 376642.53 | 178599.2 |
| C1/F_obs (ml/kg × h) | 50205.01 | 4010.1 |

Additional Formulations

Additional formulations are developed for various purposes, with the aim of obtaining different oral administration forms: sublingual drops, formulations intended for capsule packaging, and reconstituted formulations. The formulations are detailed in Table 22.

TABLE 22
Sub-lingual, capsule and reconstituted formulations

| | | | | | Reconstituted | | | |
|---|---|---|---|---|---|---|---|---|
| | Sublingual | | Formulations for | | (% wt) | | | |
| | drops (% wt) | | capsules (% wt) | | C1 before | C1, 1:10 | C2 before | C2, 1:10 |
| Ingredient | A1 | A2 | B1 | B2 | dilution | dilution | dilution | dilution |
| Polypropylene | 23-25 | 22-24 | 26-28 | 0 | 25-27 | 2.5-2.7 | 24-27 | 2.4-2.7 |
| glycol | | | | | | | | |
| PEG 400 | 23-25 | 22-24 | 16-18 | 0 | 25-27 | 2.5-2.7 | 24-27 | 2.4-2.7 |
| PRG 4000 | 0 | 0 | 10 | 10-12 | 0 | 0 | 0 | 0 |
| Cremophor | 23-25 | 22-24 | 26-28 | 40-45 | 25-27 | 2.5-2.7 | 24-27 | 2.4-2.7 |
| RH40 | | | | | | | | |
| Tripropionin or | 13-15 | 12-14 | 15-16 | 40-45 | 3-4 | 0.3-0.4 | 3-4 | 0.3-0.4 |
| tributyrin | | | | | | | | |
| Medium chain | 0 | 0 | 0 | 0 | 10-12 | 1.0-1.2 | 10-12 | 1.0-1.2 |
| triglyceride | | | | | | | | |
| Vitamin E | 0.05 | 0.05 | 0.05 | 0.05 | 0.05 | 0.005 | 0.05 | 0.005 |
| acetate | | | | | | | | |
| Peppermint | 10 | 8-10 | 0 | 0 | 3 | 0.3 | 2-3 | 0.2-0.3 |
| flavor | | | | | | | | |
| Sucralose | 0.08 | 0.6-0.8 | 0 | 0 | 0.3 | 0.03 | 0.2-0.3 | 0.02-0.03 |
| Water | 0 | 0 | 0 | 0 | 0 | 90 | 0 | 90 |
| Methylparaben | 0 | 0 | 0 | 0 | 0.6-0.9 | 0.06-0.09 | 0.6-0.9 | 0.06-0.09 |
| Propylparaben | 0 | 0 | 0 | 0 | 0.2-0.7 | 0.02-0.07 | 0.2-0.7 | 0.02-0.07 |
| CBD | 0.19-4.8 | 0.19-9.6 | 0.19-4.8 | 0.096-9.6 | 0.19-4.8 | 0.019-0.48 | 0.19-9.6 | 0.038-0.96 |
| THC | 0-0.96 | 0-1.92 | 0-0.96 | 0-0.96 | 0-0.96 | 0-0.096 | 0-0.96 | 0-0.096 |

Optimization of taste is carried out for the sublingual drops formulations by adding various flavoring agents and sweeteners. PEG4000 is added to the formulations intended for capsule packaging (see also results hereinabove) in order to increase viscosity of the formulations and minimize and/or prevent leakage of the formulation from the capsules. For the reconstituted formulations, preservatives, flavoring agents and sweeteners are added; optimization of the oil component is also carried out to prevent sedimentation post-dilution (as detailed in Table 23).

Further increase in stability of semi-solid formulations is carried out by developing formulations based on PEG4000, as shown in Table 24.

TABLE 23
Various oil components for oral reconstituted formulations

| | | | | | | | | Sedimentation | | |
|---|---|---|---|---|---|---|---|---|---|---|
| | PEG400 | CRM | PG | CBD | Vit. E | | | post dilution | Size (nm) | ZP |
| | (wt %) | (wt %) | (wt %) | (wt %) | (wt %) | TPN | MCT | (1:10) | [PDI] | (mV) |
| A | 26.59 | 26.59 | 26.59 | 5 | 0.05 | 0 | 15.19 | No sediment | 54.00 | −7.85 |
| | | | | | | | | | [0.133] | |
| B | 26.59 | 26.59 | 26.59 | 5 | 0.05 | 3.79 | 11.4 | No sediment | 125.83 | −7.88 |
| | | | | | | | | | [0.451] | |
| C | 26.59 | 26.59 | 26.59 | 5 | 0.05 | 7.6 | 7.6 | Sedimentation | 157.17 | −8.29 |
| | | | | | | | | | [0.465] | |
| D | 26.59 | 26.59 | 26.59 | 5 | 0.05 | 11.4 | 3.79 | sedimentation | 149.97 | -7.14 |
| | | | | | | | | | [0.350] | |

TABLE 24
Formulations for capsule filling

| | D1 (mg/capsule) | D2 (mg/capsule) |
|---|---|---|
| Propylene glycol | 13-168 | 0 |
| PEG400 | 8-108 | 0 |
| PEG4000 | 5-60 | 10-60 |
| Cremophor RH40 | 13-168 | 40-225 |
| TPN or TBN | 7.5-96 | 40-225 |
| Vitamin E acetate | 0.025-0.3 | 0.1-100 |
| CBD | 0.1-30 | 0.1-100 |
| THC | 0-0.5 | 0-5 |
| Filling volume (μl/capsule) | 50-600 | 100-500 |
| Capsule type | Gelatin | Gelatin, HPMC |

1-6. (canceled)

7. A cannabinoid-loaded self-emulsifying formulation comprising at least one cannabinoid, at least one oil in a content of at least 10 wt % of the formulation, at least one surfactant, and at least one structurant, adapted to form oily droplets in an aqueous diluent, said droplets having a droplet-diluent interface energy of greater than zero.

8. (canceled)

9. The formulation of claim 1, being free of water.

10-17. (canceled)

18. The formulation of claim 1, wherein said at least one structurant is selected from polyethylene glycol (PEG), propylene glycol (PG), glycerin, and combinations thereof.

19-21. (canceled)

22. The formulation of claim 1, wherein the cannabinoid is at least one of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidiorcol (CBD-C¬1), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C₄ (THCA-C4), delta-9-tetrahydrocannabinol-C4 (THCA-C4), delta-9-tetrahydrocannabivarinic acid (THCVA), delta-9-tetrahydrocannabivarin (THCV), delta-9-tetrahydrocannabiorcolic acid (THCA-C1), delta-9-tetrahydrocannabiorcol (THC-C1), delta-7-cis-iso-tetrahydrocannabivarin, delta-8-tetrahydrocannabinolic acid A (Δ8-THCA), delta-8-tetrahydrocannabinol (Δ8-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahtdro-7-hydroxy-α-α-2-trimethyl-9-n-propyl-2,6-methano-2H-1-benzoxocin-5-methanol (OH-iso-HHCV), cannabiripsol (CBR), trihydroxy-delta-9-tetrahydroxycannabinol (triOH-THC).

23-28. (canceled)

29. A cannabinoid-loaded self-emulsifying formulation comprising at least one cannabinoid, at least one oil in a content of at least 10 wt % of the formulation, at least one surfactant, and at least one structurant, said at least one oil comprising tripropionin or tributyrin.

30. (canceled)

31. A self-emulsifying formulation for oral delivery of at least one lipophilic compound, the formulation comprising at least one lipophilic compound, at least one oil in a content of at least 10 wt % of the formulation, at least one surfactant, and at least one structurant.

32. The formulation of claim 31, wherein said lipophilic compound is selected from at least one cannabinoid, CB1 receptor blockers with molecular weights ranging from 150 to 1200 Da, oxaliplatin palmitate acetate (OPA), cyclosporine A, a vitamin, an anti-oxidant, a lipid, a hormone, an antibiotic agent, a prophylactic agent, a small molecule of a molecular weight of less than about 1,000 Da or less than about 500 D, an analgesic or anti-inflammatory agent; an anthelmintic agent; an anti-arrhythmic agent; an anti-bacterial agent; an anti-coagulant; an anti-depressant; an antidiabetic; an anti-epileptic; an anti-fungal agent; an anti-gout agent; an anti-hypertensive agent; an anti-malarial agent; an anti-migraine agent; an anti-, muscarinic agent; an anti-neuroplastic agent or immunosuppressant; an anti-protazoal agent; an anti-thyroid agent; an alixiolytic, sedative, hypnotic or neuroleptic agent; a beta-blocker; a cardiac inotropic agent; a corticosteroid; a diuretic agent; an anti-Parkinsonian agent; a gastro-intestinal agent; an histamine H1-receptor antagonist; a lipid regulating agent; a nitrate or anti-anginal agent; a nutritional agent; an HIV protease inhibitor; an opioid analgesic; capsaicin a sex hormone; a cytotoxic agent; and a stimulant agent, and any combination thereof.

33. (canceled)

34. The formulation of claim 32, wherein said at least one lipophilic compound is at least one CB1 receptor blocker with molecular weight ranging from 150 to 1200 Da.

35-38. (canceled)

39. A pharmaceutical composition comprising the formulation of claim 1.

40. The pharmaceutical composition of claim 39, further comprising a pharmaceutically acceptable carrier.

41. The pharmaceutical composition of claim 39, further comprising an aqueous diluent.

42. The pharmaceutical composition of claim 41, wherein the aqueous diluent is selected from water, saline, dextrose solution, water/alcohol mixtures, sweetener-containing aqueous solutions, flavor-containing aqueous solutions, and an isotonic solution.

43. The pharmaceutical composition of claim 41, wherein the composition is in the form of a nanoemulsion, comprising oily droplets of said formulation having a mean diameter of at least 100 nm, dispersed in a continuous phase constituted by the aqueous diluent.

44. The pharmaceutical composition of claim 43, wherein the mean diameter of the droplets is between about 100 nm and 800 nm.

45. A unit dosage form for oral delivery of at least one cannabinoid, the unit dosage form comprising the formulation of claim 1.

46. (canceled)

47. A kit comprising a formulation according to claim 1 and an aqueous diluent.

48-50. (canceled)

51. A method of treating a subject suffering from a condition or a disorder, the method comprising orally administering to the subject an effective amount of the formulation of claim 1.
